(19) United States
(12) Patent Application Publication
(10) Pub. No.: US 2009/0123471 A1
(43) Pub. Date: May 14, 2009

(54) T-Cadherin antigen arrays and uses thereof

(75) Inventors: Martin F. Bachmann, Seuzach (CH); Philippe Saudan, Pfungen (CH); Klaus Dietmeier, Zurich (CH); Alain Tissot, Zurich (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: Cytos Biotechnology AG, Zurich-Schlieren (CH)

(21) Appl. No.: 11/666,497

(22) PCT Filed: Oct. 31, 2005
(86) PCT No.: PCT/EP05/55668

§ 371 Date: Apr. 27, 2007

Related U.S. Application Data
(60) Provisional application No. 60/623,048, filed on Oct. 29, 2004.

(51) Int. Cl. A61K 39/395; C07K 14/00; A61K 39/00; A61K 38/16

(52) U.S. Cl. 424/139.1; 530/350; 424/185.1; 514/12

(57) ABSTRACT

The present invention is in the fields of medicine, public health, immunology, molecular biology and virology. The present invention provides, inter alia, a composition comprising a virus-like particle (VLP) and at least one antigen, wherein said antigen is a T-cadherin domain protein, a combination of any T-cadherin domain proteins, a T-cadherin domain fragment or a combination of any T-cadherin domain fragments, linked to the VLP respectively. The invention also provides a method for producing the aforesaid composition. The compositions of this invention are useful in the production of vaccines, in particular, for the prevention and/or treatment of T-cadherin related disease, and hereby, in particular, by inducing efficient immune responses, in particular antibody responses. Furthermore, the compositions of the invention are particularly useful to efficiently induce self-specific immune responses within the indicated context.

### BACKGROUND OF THE INVENTION

1. Field of the Invention

The present invention is in the fields of medicine, public health, immunology, molecular biology and virology. The present invention provides, inter alia, a composition comprising a virus-like particle (VLP) and at least one antigen, wherein said antigen is a T-cadherin domain protein, a combination of any T-cadherin domain proteins, a T-cadherin domain fragment or a combination of any T-cadherin domain fragments, linked to the VLP respectively.

The invention also provides a method for producing the aforesaid composition. The compositions of this invention are useful in the production of vaccines, in particular, for the prevention and/or treatment of T-cadherin related diseases, and hereby, in particular, by inducing efficient immune responses, in particular antibody responses. Furthermore, the compositions of the invention are particularly useful to efficiently induce self-specific immune responses within the indicated context.

2. Related Art

T-cadherin, also known as H-cadherin and cadherin 13, contains five extracellular domains also referred to as cadherin repeats (CADs). However, it lacks both the transmembrane domain and the cytoplasmic domain found in other members of the cadherin family. It is anchored to the membrane via a glycosylphosphatidylinositol (GPI) which is attached during the processing in the endoplasmatic reticulum (Ranscht, B. and Dours-Zimmerman M. T., (1991), Neuron, 7, 391-402). T-cadherin has been shown to be able to mediate weak homophilic interactions (Vestal, D. J., and Ranscht, B. (1992) J. Cell Biol., 119, 451-461). The established function of T-cadherin is its participation in the regulation of neuron growth during embryogenesis. During formation of chick embryo hind limbs, the outgrowing axons avoid those regions where T-cadherin is expressed (Fredette, B. J., and Ranscht, B. (1994) J. Neurosci., 14, 7331-7346).

More recently T-cadherin has been reported to suppress tumor growth in vivo. In fact, over-expression of T cadherin in tumor cells was shown to decrease the proliferative and invasive activities both in vitro (Lee, S. W. (1996) Nat. Med., 2, 776-782) and in vivo (Lee, S. W. (1998) Carcinogenesis, 19, 1157-1159).

Studies revealed high level expression of T cadherin in the vasculature. T-cadherin was found to be expressed in all layers of the vascular wall, with the highest level of expression on the smooth muscle and pericyte-like cells in the proteoglycan layer of the intima and the vasa vasorum. Moreover, like other cell adhesion molecules such as VCAM-1 on ECs, β1-integrins and VCAM-1 on small muscle cells and E-cadherin on foam cells (Hedin, U. et al., (1988), J Cell Biol, 107:307-319, Boudreau, N. et al., (1991), Dev Biol 143:235-247, Li, H. et al. (1993), Arterioscler Thromb 13:197-204, O'Brien, K. D., et al (1993), J Clin Invest 92:945-951, Bobryshev, Y. V. et al (1998), Cardiovasc Res 40:191-205), increased expression of T cadherin has also been shown to correlate with the pathogenesis of the atherosclerotic lesion (Ivanov, D. et al., (2001) Histochem Cell Biol., 115: 231-242).

### SUMMARY OF THE INVENTION

We have, now, surprisingly found that the inventive compositions and vaccines, respectively, comprising at least one T-caherin of the invention linked to a VLP of the invention are capable of inducing strong immune responses, in particular strong antibody responses, leading to high antibody titer against the self-antigen T-cadherin. Moreover, we have surprisingly found that inventive compositions and vaccines, respectively, are capable of inducing strong immune responses, in particular strong antibody responses, for the treatment of atherosclerosis as indicated by administration of the inventive compositions and vaccines, respectively, in Apoe^{-/-} mice, which is a mouse model for atherosclerosis. The atherosclerosis condition has been significantly improved in mice received the inventive compositions and vaccines, respectively, as compared to mice used as negative control. This indicates that the immune responses, in particular the antibodies generated by the inventive compositions and vaccines, respectively, are thus capable of specifically recognizing T-cadherin in vivo and interfere with its function. This indicates that T-cadherin can be used as a valuable drug target, and hereby in particular in treating atherosclerosis.

Thus, in a first aspect, the present invention provides a composition which comprises (a) a virus-like particle (VLP), with at least one first attachment site; and (b) at least one antigen with at least one second attachment site, wherein said at least one antigen is a T-cadherin domain protein, a combination of T-cadherin domain proteins, a T-cadherin domain fragment or a combination of T-cadherin domain fragments, wherein (a) and (b) are linked through the first and the second attachment sites, preferably to form an ordered and repetitive antigen array.

In one further preferred embodiment, the VLP of the invention is recombinantly produced in a host and said VLP is essentially free of host RNA or host DNA, preferably host nucleic acid. It is advantageous to reduce, or preferably to eliminate, the amount of host RNA or host DNA, preferably nucleic acid to avoid unwanted T cell responses as well as other unwanted side effects, such as fever.

In one preferred embodiment, the at least one antigen is a T-cadherin domain protein. In a further preferred embodiment, the T-cadherin domain protein is a T-cadherin domain 1 protein. In one alternatively preferred embodiment, the at least one antigen is a combination of T-cadherin domain 1 protein and T-cadherin domain 2 protein.

In one preferred embodiment, the at least one antigen is a T-cadherin domain fragment, wherein the domain fragment comprises at least one antigenic site of T-cadherin. While ensuring a strong and protective immune response, in particular an antibody response, the use of T-cadherin domain fragments for the present invention may reduce a possible induction of self-specific cytotoxic T cell responses and may reduce the production cost of the inventive compositions and vaccines, respectively.

In another aspect, the present invention provides a vaccine composition comprising VLP-T-cadherin conjugates. Furthermore, the present invention provides a method to administering the vaccine composition to a human or an animal, preferably a mammal. The vaccine of the present invention is capable of inducing a strong immune response, in particular an antibody response, without the presence of any adjuvant. Thus, in one preferred embodiment, the vaccine is devoid of any adjuvant. The avoidance of using adjuvant may reduce a possible occurrence of unwanted inflammatory T cell responses.

In a further aspect, the present invention provides for a pharmaceutical composition comprising the inventive composition and an acceptable pharmaceutical carrier.

In a still further aspect, the present invention provides for a method of treating a diseases comprising administering the inventive composition or the inventive vaccine composition to a animal or to a human, wherein said disease is selected from form the group consisting of coronary artery disease, atherosclerosis, obesity, type I diabetes, type II diabetes and cancer.

In again a further aspect, the present invention provides for a method of producing the composition of the invention comprising (a) providing a VLP with at least one first attachment site; (b) providing at least one antigen, wherein said antigen is a T-cadherin domain protein, a combination of T-cadherin domain proteins, a T-cadherin domain fragment or a combination of T-cadherin domain fragments, with at least one second attachment site; and (c) combining said VLP and said at least one antigen to produce said composition, wherein said at least one antigen and said VLP are linked through said at least one first and said at least one second attachment sites.

In one aspect, the invention provides a method of treating a disease in an animal or a human comprising administering at least one substance to said animal or human, wherein said substance is characterized by being capable of binding to T-cadherin and wherein said disease is selected form the group consisting of coronary artery disease, atherosclerosis, obesity, type I diabetes, type II diabetes and cancer, preferably in cancer in which angiogenesis plays an important role.

In one preferred embodiment, the substance is an antibody against T-cadherin, wherein said antibody is preferably a monoclonal antibody.

### BRIEF DESCRIPTION OF THE FIGURES

FIG. 1 shows the coupling of T-cadherin domain protein CAD1 (SEQ ID NO:46) to the prior art Qβ VLP on a reducing SDS-PAGE gel. T-Lane M is the molecular marker; Lane 1 is the derivatized Qβ monomer; Lane 2 is the T-cadherin fragment 1-15 GC coupled to the Qβ monomer; Lane 3 is the T-cadherin fragment 31-42 GC coupled to the Qβ monomer. Coupling bands corresponding to one, two or three CAD1 coupled per Qβ subunit are indicated.

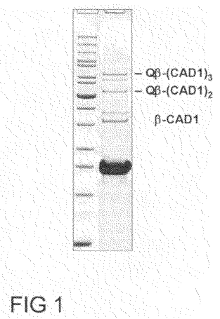

FIG. 2 shows the quantification and statistical analysis of the atherosclerotic plaque load in Apoe^{-/-} mice received prior art Qβ coupled to CAD1 or Qβ only groups. Data are plotted in a box and whiskers graph. The middle line of the box corresponds to the median, while the upper boundary of the box is the 75^{th} percentile and the bottom boundary of the box is the 25^{th} percentile. The whiskers extend to the highest and lowest value respectively.

### DETAILED DESCRIPTION OF THE INVENTION

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

Antigenic site: The term “antigenic site” and the term “antigenic epitope”, which are used herein interchangeably, refer to continuous or discontinuous portions of a polypeptide, which can be bound immunospecifically by an antibody or by a T-cell receptor within the context of an MHC molecule. Immunospecific binding excludes non-specific binding but does not necessarily exclude cross-reactivity. Antigenic site typically comprise 5-10 amino acids in a spatial conformation which is unique to the antigenic site.

Associated: The term “associated” (or its noun association) as used herein refers to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds.

Attachment Site, First: As used herein, the phrase “first attachment site” refers to an element which is naturally occurring with the VLP or which is artificially added to the VLP, and to which the second attachment site may be linked. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid such as lysine. The first attachment site is located, typically on the surface, and preferably on the outer surface of the VLP. Multiple first attachment sites are present on the surface, preferably on the outer surface of virus-like particle, typically in a repetitive configuration. In a preferred embodiment the first attachment site is associated with the VLP, through at least one covalent bond, preferably through at least one peptide bond.

Attachment Site, Second: As used herein, the phrase “second attachment site” refers to an element which is naturally occurring with or which is artificially added to the T-cadherin of the invention and to which the first attachment site may be linked. The second attachment site of T-cadherin of the invention may be a protein, a polypeptide, a peptide, an amino acid, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is the sulfhydryl group, preferably of an amino acid cysteine. The term “T-cadherin domain protein with at least one second attachment site”, “T-cadherin domain fragment with at least one second attachment site” or “a combination of T-cadherin domain proteins with at least one second attachment site” or “a combination of T-cadherin domain fragments with at least one second attachment site” refers, therefore, to a construct comprising the T-cadherin of the invention and at least one second attachment site. However, in particular for a second attachment site, which is not naturally occurring within the T-cadherin domain protein or the T-cadherin domain fragment, such a construct typically and preferably further comprises a “linker”. In one preferred embodiment the second attachment site is associated with the T-cadherin of the invention through at least one covalent bond, preferably through at least one peptide bond. In yet another preferred embodiment, the second attachment site is artificially added to the T-cadherin of the invention through an amino acid linker, wherein preferably said amino acid linker comprises a cysteine, by protein fusion.

Bound: As used herein, the term “bound” refers to binding that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term also includes the enclosement, or partial enclosement, of a substance. The term “bound” is broader than and includes terms such as “coupled,” “fused,” “enclosed”, “packaged” and “attached.” For example, the polyanionic macromolecule such as the polyglutamic acid can be, and typically and preferably is, enclosed or packaged by the VLP, typically and preferably without the existence of an actual covalent binding.

Coat protein: The term “coat protein” and the interchangeably used term “capsid protein” within this application, refers to a viral protein, which is capable of being incorporated into a virus capsid or a VLP. Typically and preferably the term “coat protein” refers to the coat protein encoded by the genome of a virus, preferably an RNA bacteriophage or by the genome of a virus, preferably a variant of an RNA bacteriophage. More preferably and by way of example, the term “coat protein of AP205” refers to SEQ ID NO:14 or the amino acid sequence, wherein the first methionine is cleaved from SEQ ID NO:14. More preferably and by way of example, the term “coat protein of Qβ” refers to SEQ ID NO:1 (“Qβ CP”) and SEQ ID NO:2 (A1), with or without the methione at the N-terminus. The capsid of bacteriophage Qβ is composed mainly of the Qβ CP, with a minor content of the A1 protein.

Linked: The term “linked” (or its noun: linkage) as used herein, refers to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, amide, peptide, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide bond(s). The term “linked” as used herein, however, shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Linker: A “linker”, as used herein, either associates the second attachment site with T-cadherin of the invention or already comprises, essentially consists of, or consists of the second attachment site. Preferably, a “linker”, as used herein, already comprises the second attachment site, typically and preferably—but not necessarily—as one amino acid residue, preferably as a cysteine residue. A “linker” as used herein is also termed “amino acid linker”, in particular when a linker according to the invention contains at least one amino acid residue. Thus, the terms “linker” and “amino acid linker” are interchangeably used herein. However, this does not imply that such a linker consists exclusively of amino acid residues, even if a linker consisting of amino acid residues is a preferred embodiment of the present invention. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a suffhydryl group or a cysteine residue and such molecules are, therefore, also encompassed within this invention. Further linkers useful for the present invention are molecules comprising a C1-C6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the T-cadherin of the invention is preferably by way of at least one covalent bond, more preferably by way of at least one peptide bond.

Ordered and repetitive antigen array: As used herein, the term “ordered and repetitive antigen array” generally refers to a repeating pattern of antigen, characterized by a typically and preferably high order of uniformity in spacial arrangement of the antigens with respect to virus-like particle, respectively. In one embodiment of the invention, the repeating pattern may be a geometric pattern. Certain embodiments of the invention, such as VLP of RNA phages, are typical and preferred examples of suitable ordered and repetitive antigen arrays which, moreover, possess strictly repetitive paracrystalline orders of antigens, preferably with spacings of 1 to 30 nanometers, preferably 2 to 15 nanometers, even more preferably 2 to 10 nanometers, even again more preferably 2 to 8 nanometers, and further more preferably 1.6 to 7 nanometers.

Packaged: The term “packaged” as used herein refers to the state of a polyanionic macromolecule in relation to the VLP. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule can be enclosed by the VLP without the existence of an actual binding, in particular of a covalent binding. In preferred embodiments, the at least one polyanionic macromolecule is packaged inside the VLP, most preferably in a non-covalent manner.

Polypeptide: The term “polypeptide” as used herein refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. Post-translational modifications of the polypeptide, for example, glycosylations, acetylations, phosphorylations, and the like are also encompassed.

T-cadherin of the invention: The term “T-cadherin of the invention” as used herein, refers to at least one T-cadherin domain protein, any combination of at least two, preferably two, T-cadherin domain proteins, at least one T-cadherin domain fragment or any combination of at least two, preferably T-cadherin domain fragments as defined herein or any combination thereof.

T-cadherin: The term “T-cadherin”, as used herein, refers to the human T-cadherin, which is also known as H-cadherin and Cadherin 13 in the prior art as well as orthologs of human T-cadherin from other animals, preferably from mammals, even more preferably from dog, mouse, rat and cat. The term “ortholog” denotes a polypeptide obtained from one species that is the functional counterpart of a polypeptide from a different species. Sequence differences among orthologs are the result of speciation.

T-cadherin domain: The term “T-cadherin domain”, as used herein, refers to any one of the extracellular domains comprised by a T-cadherin. More specifically, the term “extracellular domain 1”, as used herein, refers to the first extracellular domain from the amino terminus of T-cadherin. Correspondingly, the terms “extracellular domain 2”, “extracellular domain 3”, “extracellular domain 4”, or “extracellular domain 5”, as used herein, refer to the second, third, fourth or fifth extracellular domain from the amino terminus of T-cadherin. The aforementioned numbering of the extracellular domains of T-cadherin is in agreement with at least most of the numbering and nomenclature, respectively, used in the art. The terms “extracellular domain 1” and “domain 1”, the terms “extracellular domain 2” and “domain 2”, the terms “extracellular domain 3” and “domain 3”, the terms “extracellular domain 4” and “domain 4”, as well as the terms “extracellular domain 5” and “domain 5”, are interchangeably used in this application.

T-cadherin domain protein: The term “T-cadherin domain protein”, as used herein, should encompass any polypeptide comprising, or alternatively or preferably consisting of, any one of the extracellular domains comprised by a T-cadherin, i.e. any T-cadherin domain, as well as 60, preferably 70, more preferably 80, more preferably 85, more preferably 90, more preferably 95, more preferably 100, and when applicable, more preferably 105, more preferably 110, more preferably 115 amino acid sequence in the middle of the sequence of any one of the extracellular domains comprised by a T-cadherin. Furthermore, the term “T-cadherin domain protein” should encompass any polypeptide comprising, or alternatively or preferably consisting of, an amino acid sequence which is 70%, preferably 80%, more preferably 90%, more preferably 95%, more preferably 97%, more preferably 99% identical to the amino acid sequence of a “T-cadherin domain protein”, as defined hereabove. The term “T-cadherin domain protein” as used herein should furthermore encompass post-translational modifications including but not limited to glycosylations, acetylations, phosphorylations of the T-cadherin domain protein as defined above. Preferably the T-cadherin domain protein, as defined herein, consists of at most 500 amino acids in length, preferably 400, and even more preferably of at most 300, more preferably 250, more preferably 200 amino acids in length. Typically and preferably, T-cadherin domain protein is capable of inducing in vivo the production of antibody specifically binding to the corresponding T-cadherin domain, as verified by, for example ELISA.

More specifically, the term “T-cadherin extracellular domain 1 protein”, as used herein, refers to a T-cadherin domain protein, as defined hereabove, and deriving from the extracellular domain 1 of T-cadherin. Correspondingly, the terms “T-cadherin extracellular domain 2 protein”, “T-cadherin extracellular domain 3 protein”, “T-cadherin extracellular domain 4 protein”, or “T-cadberin extracellular domain 5 protein”, as used herein, refer to a T-cadherin domain protein, as defined hereabove, and deriving from the extracellular domain 2, 3, 4 or 5 of T-cadherin.

For the determination of the amino acid sequence being in the middle of the aforementioned sequences as well as for the below mentioned sequences, the term “middle”, as used herein, should refer to parts of the mentioned sequences, which cut off the exact same numbers of amino acids at the N-terminus and at the C-terminus from the mentioned sequences. In case of the mentioned sequences having odd numbers of amino acids, the term “middle”, as used herein, should refer to parts of the mentioned sequences which cut off exactly one amino acid less at the N-terminus as compared to the C-terminus from the mentioned sequences as well as to parts of the mentioned sequences which cut off exactly one amino acid less at the C-terminus as compared to the N-terminus from the mentioned sequences.

The term “human T-cadherin domain protein”, the term “mouse T-cadherin domain protein”, the term “cat T-cadherin domain protein” or the term “dog T-cadherin domain protein” or the like from other animals is a species of the term “T-cadherin domain protein”. Likewise, the term “human T-cadherin domain 1 protein” or the term “human T-cadherin domain 2 protein” or the like, is a species of the term “T-cadherin domain 1 protein” or the term “T-cadherin domain 2 protein” or the like. The extracellular domain 1, 2, 3, 4 or 5 of human T-cadherin is given in SEQ ID NO: 40, 41, 42, 43 or 44. The extracellular domain 1, 2, 3, 4 or 5 of mouse T-cadherin is given in SEQ ID NO: 24, 25, 26, 27 or 28.

Combination of any T-cadherin domain proteins: the term “combination of any T-cadherin domain proteins”, as used herein, should encompass any polypeptide comprising, or alternatively or preferably consisting of, at least two, and hereby preferably two, identical or different, and hereby preferably different, T-cadherin domain proteins, wherein said at least two, and preferably two, T-cadherin domain proteins are in a discontinuous order or preferably in a consecutive order as in the native T-cadherin. Preferred embodiments of a combination of at least two T-cadherin domain proteins are a combination of T-cadherin domain 1 protein and domain 2 protein, a combination of T-cadherin domain 2 protein and domain 3 protein, or a combination of T-cadherin domain 1 protein and domain 3 protein. In a preferred embodiment, said polypeptide further comprises spacer sequence between T-cadherin domain proteins.

T-cadherin domain fragment: The term “T-cadherin domain fragment” as used herein should encompass any polypeptide comprising, or alternatively or preferably consisting of, at least 5, 6, 7, 8, 9, 10, 11, 12, 17, 18, 19, 20, 25 or 30 contiguous amino acids of a T-cadherin domain protein as defined herein as well as any polypeptide having more than 65%, preferably more than 80%, more preferably more than 90% and even more preferably more than 95% amino acid sequence identity thereto. Preferred embodiments of T-cadherin domain fragments are truncation forms of T-cadherin domain protein. Preferred embodiments of T-cadherin domain fragments have no more than 60, preferably no more than 50, more preferably no more than 30, still more preferably no more than 20 amino acids. Preferably, a T-cadherin domain fragment is capable of inducing the production of antibody in vivo, which specifically binds to the corresponding T-cadherin domain.

Combination of any T-cadherin protein domain fragments: The term “combination of any T-cadherin protein domain fragments”, as used herein, should encompass any polypeptide comprising, or alternatively or preferably consisting of, at least two, and preferably four, more preferably three, still more preferably two, T-cadherin protein domain fragments. In a preferred embodiment, said polypeptide further comprises spacer sequence between T-cadherin domain fragments.

The amino acid sequence identity of polypeptides can be determined conventionally using known computer programs such as the Bestfit program. When using Bestfit or any other sequence alignment program, preferably using Bestfit, to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, polypeptides or a fragment thereof disclosed in this invention.

Virus particle: The term “virus particle” as used herein refers to the morphological form of a virus. In some virus types it comprises a genome surrounded by a protein capsid; others have additional structures (e.g., envelopes, tails, etc.).

Virus-like particle (VLP), as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. Preferably a virus-like particle in accordance with the invention is non-replicative and/or non-infectious since it lacks all or part of the viral genome or genome function. In one embodiment, a virus-like particle is a virus particle, in which the viral genome has been physically or chemically inactivated. Typically and more preferably a virus-like particle lacks all or part of the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, preferably RNA-phage. The terms “viral capsid” or “capsid”, refer to a macromolecular assembly composed of viral protein subunits. Typically, there are 60, 120, 180, 240, 300, 360 and more than 360 viral protein subunits. Typically and preferably, the interactions of these subunits lead to the formation of viral capsid or viral-capsid like structure with an inherent repetitive organization, wherein said structure is, typically, spherical or tubular.

Virus-like particle of a RNA phage: As used herein, the term “virus-like particle of a RNA phage” refers to a virus-like particle comprising, or preferably consisting essentially of or consisting of coat proteins, mutants or fragments thereof, of a RNA phage. In addition, virus-like particle of a RNA phage resembling the structure of a RNA phage, being non replicative and/or non-infectious, and lacking at least the gene or genes encoding for the replication machinery of the RNA phage, and typically also lacking the gene or genes encoding the protein or proteins responsible for viral attachment to or entry into the host. This definition should, however, also encompass virus-like particles of RNA phages, in which the aforementioned gene or genes are still present but inactive, and, therefore, also leading to non-replicative and/or non-infectious virus-like particles of a RNA phage. Preferred VLPs derived from RNA-phages exhibit icosahedral symmetry and consist of 180 subunits. Within this present disclosure the term “subunit” and “monomer” are interexchangeably and equivalently used within this context. In this application, the term “RNA-phage” and the term “RNA-bacteriophage” are interchangeably used.

Within this application, antibodies are defined to be specifically binding if they bind to the antigen with a binding affinity (Ka) of 10⁶ M⁻¹ or greater, preferably 10⁷ M⁻¹ or greater, more preferably 10⁸ M⁻¹ or greater, and most preferably 10⁹ M⁻¹ or greater. The affinity of an antibody can be readily determined by one of ordinary skill in the art (for example, by Scatchard analysis.)

One, a, or an: when the terms “one”, “a”, or “an” are used in this disclosure, they mean “at least one” or “one or more” unless otherwise indicated.

This invention provides compositions and methods for enhancing immune responses against T-cadherin in an animal or in human. Compositions of the invention comprise (a) a virus-like particle (VLP) with at least one first attachment site; and (b) at least one antigen with at least one second attachment site, wherein said at least one antigen is a T-cadherin domain protein, a combination of T-cadherin domain proteins, a T-cadherin domain fragment or a combination of T-cadherin domain fragments, wherein (a) and (b) are linked through said at least one first and said at least one second attachment site. Preferably, the T-cadherin of the invention is linked to the VLP, so as to form an ordered and repetitive antigen-VLP array. In preferred embodiments of the invention, at least 30, more preferably at least 60, again more preferably at least 120 and further more preferably at least 180 T-cadherin of the invention are linked to the VLP.

Any virus known in the art having an ordered and repetitive structure may be selected as a VLP of the invention. Illustrative DNA or RNA viruses, the coat or capsid protein of which can be used for the preparation of VLPs have been disclosed in WO 2004/009124 on page 25, line 10-21, on page 26, line 11-28, and on page 28, line 4 to page 31, line 4. These disclosures are incorporated herein by way of reference.

Virus or virus-like particle can be produced and purified from virus-infected cell culture. The resulting virus or virus-like particle for vaccine purpose needs to be devoid of virulence. Avirulent virus or virus-like particle may be generated by chemical and/or physical inactivation, such as UV irradiation, formaldehyde treatment. Alternatively, the genome of the virus may be genetically manipulated by mutations or deletions to render the virus replication incompetent.

In one preferred embodiment, the VLP is a recombinant VLP. Recombinant VLP as disclosed herein, refers to a VLP which is prepared by a process comprising at least one step of DNA recombination technology. Almost all commonly known viruses have been sequenced and are readily available to the public. The gene encoding the coat protein can be easily identified by a skilled artisan. Typically, the coat protein gene can be cloned by standard methods into an expression vector and expressed in a vector-suitable host. The VLP, resulted from the self assembly of the expressed coat protein can be recovered and further purified by methods commonly known in the art. Examples have been disclosed in WO02/056905 and are herein incorporated by way of reference: Suitable host cells for virus-like particle production on page 29, line 37, to page 30, line 12; methods for introducing polynucleotide vectors into host cells on page 30, lines 13-27 and mammalian cells as recombinant host cells for the production of virus-like particles on page 30, lines 28-35.

In one preferred embodiment, the virus-like particle comprises, or alternatively consists of, recombinant proteins, mutants or fragments thereof, of a virus selected form the group consisting of: a) RNA phages; b) bacteriophages; c) Hepatitis B virus, preferably its capsid protein (Ulrich, et al., Virus Res. 50:141-182 (1998)) or its surface protein (WO 92/11291); d) measles virus (Warnes, et al., Gene 160:173-178 (1995)); e) Sindbis virus; f) rotavirus (U.S. Pat. No. 5,071,651 and U.S. Pat. No. 5,374,426); g) foot-and-mouth-disease virus (Twomey, et al., Vaccine 13:1603 1610, (1995)); h) Norwalk virus (Jiang, X., et al., Science 250:1580 1583 (1990); Matsui, S. M., et al., J. Clin. Invest. 87:1456 1461 (1991)); i) Alphavirus; j) retrovirus, preferably its GAG protein (WO 96/30523); k) retrotransposon Ty, preferably the protein p1; 1) human Papilloma virus (WO 98/15631); m) Polyoma virus; n) Tobacco mosaic virus; and o) Flock House Virus.

In one preferred embodiment, the VLP comprises, or consists of, more than one amino acid sequence, preferably two amino acid sequences, of the recombinant proteins, mutants or fragments thereof. VLP comprises or consists of more than one amino acid sequence is referred, in this application, as mosaic VLP.

The term “fragment of a recombinant protein” or the term “fragment of a coat protein”, as used herein, is defined as a polypeptide, which is of at least 70%, preferably at least 80%, more preferably at least 90%, even more preferably at least 95% the length of the wild-type recombinant protein, or coat protein, respectively and which preferably retains the capability of forming VLP. Preferably the fragment is obtained by at least one internal deletion, at least one truncation or at least one combination thereof. The term “fragment of a recombinant protein” or “fragment of a coat protein” shall further encompass polypeptide, which has at least 80%, preferably 90%, even more preferably 95% amino acid sequence identity with the “fragment of a recombinant protein” or “fragment of a coat protein”, respectively, as defined above and which is preferably capable of assembling into a virus-like particle.

The term “mutant recombinant protein” or the term “mutant of a recombinant protein” as interchangeably used in this invention, or the term “mutant coat protein” or the term “mutant of a coat protein”, as interchangeably used in this invention, refers to a polypeptide having an amino acid sequence derived from the wild type recombinant protein, or coat protein, respectively, wherein the amino acid sequence is at least 80%, preferably at least 85%, 90%, 95%, 97%, or 99% identical to the wild type sequence and preferably retains the ability to assemble into a VLP.

Assembly of the fragment or mutant of recombinant protein or coat protein into a VLP may be tested, as one skilled in the art would appreciate by expressing the protein in E. coli, optionally purifying the capsids by gel filtration from cell lysate, and analysing the capsid formation in an immunodiffusion assay (Ouchterlony test) or by Electron Microscopy (EM) (Kozlovska, T. M. et al., Gene 137:133-37 (1993)). Immunodiffusion assays and EM may be directly performed on cell lysate.

In one preferred embodiment, the virus-like particle of the invention is of Hepatitis B virus. The preparation of Hepatitis B virus-like particles have been disclosed, inter alia, in WO 00/32227, WO 01/85208 and in WO 01/056905. All three documents are explicitly incorporated herein by way of reference. Other variants of HBcAg suitable for use in the practice of the present invention have been disclosed in page 34-39 WO 01/056905.

In one further preferred embodiments of the invention, a lysine residue is introduced into the HBcAg polypeptide, to mediate the linking of T-cadherin of the invention to the VLP of HBcAg. In preferred embodiments, VLPs and compositions of the invention are prepared using a HBcAg comprising, or alternatively consisting of, amino acids 1-144, or 1-149, 1-185 of SEQ ID NO:20, which is modified so that the amino acids at positions 79 and 80 are replaced with a peptide having the amino acid sequence of Gly-Gly-Lys-Gly-Gly. This modification changes the SEQ ID NO:20 to SEQ ID NO:21. In further preferred embodiments, the cystcine residues at positions 48 and 110 of SEQ ID NO:21, or its corresponding fragments, preferably 1-144 or 1-149, are mutated to serine. The invention further includes compositions comprising Hepatitis B core protein mutants having above noted corresponding amino acid alterations. The invention further includes compositions and vaccines, respectively, comprising HBcAg polypeptides which comprise, or alternatively consist of, amino acid sequences which are at least 80%, 85%, 90%, 95%, 97% or 99% identical to SEQ ID NO:21.

In another embodiment of the invention, the virus-like particle is a recombinant alphavirus, and more specifically, a recombinant Sindbis virus. Alphaviruses are positive stranded RNA viruses that replicate their genomic RNA entirely in the cytoplasm of the infected ccli without a DNA intermediate (Strauss, J. and Strauss, E., Microbiol. Rev. 58:491-562 (1994)). Several members of the alphavirus family, Sindbis (Schlesinger, S., Trends Biotechnol. 11:18-22 (1993)), Semliki Forest Virus (SFV) (Liljeström, P. & Garoff, H., Bio/Technology 9:1356-1361 (1991)) and others (Davis, N. L. et al., Virology 171:189-204 (1989)), have received considerable attention for use as virus-based expression vectors for a variety of different proteins (Lundstrom, K., Curr. Opin. Biotechnol. 8:578-582 (1997)) and as candidates for vaccine development.

In preferred embodiments of the invention, the virus-like particle of the invention comprises, consists essentially of, or alternatively consists of, recombinant coat proteins, mutants or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7 and m) bacteriophage AP205.

In one preferred embodiment of the invention, the composition comprises coat protein, mutants or fragments thereof, of RNA phages, wherein the coat protein has amino acid sequence selected from the group consisting of: (a) SEQ ID NO:1, referring to Qβ CP; (b) a mixture of SEQ ID NO:1 and SEQ ID NO:2 (referring to Qβ A protein); (c) SEQ ID NO:3; (d) SEQ ID NO:4; (e) SEQ ID NO:5; (f) SEQ ID NO:6, (g) a mixture of SEQ ID NO:6 and SEQ ID NO:7; (h) SEQ ID NO:8; (i) SEQ ID NO:9; (j) SEQ ID NO:10; (k) SEQ ID NO:11; (l) SEQ ID NO:12; (m) SEQ ID NO:13; and (n) SEQ ID NO:14. Generally the coat protein mentioned above is capable of assembly into VLP with or without the presence of the N-terminal methionine.

In one preferred embodiment of the invention, the VLP is a mosaic VLP comprising or alternatively consisting of more than one amino acid sequence, preferably two amino acid sequences, of coat proteins, mutants or fragments thereof, of a RNA phage.

In one very preferred embodiment, the VLP comprises or alternatively consists of two different coat proteins of a RNA phage, said two coat proteins have an amino acid sequence of SEQ ID NO: 1 and SEQ ID NO:2, or of SEQ ID NO:6 and SEQ ID NO:7.

In preferred embodiments of the present invention, the virus-like particle of the invention comprises, or alternatively consists essentially of, or alternatively consists of recombinant coat proteins, mutants or fragments thereof, of the RNA-bacteriophage Qβ, fr, AP205 or GA.

In one preferred embodiment, the VLP of the invention is a VLP of RNA-phage Qβ. The capsid or virus-like particle of Qβ showed an icosahedral phage-like capsid structure with a diameter of 25 nm and T=3 quasi symmetry. The capsid contains 180 copies of the coat protein, which are linked in covalent pentamers and hexamers by disulfide bridges (Golmohammadi, R. et al., Structure 4:543-5554 (1996)), leading to a remarkable stability of the Qβ capsid. Capsids or VLPs made from recombinant Qβ coat protein may contain, however, subunits not linked via disulfide bonds to other subunits within the capsid, or incompletely linked. The capsid or VLP of Qβ shows unusual resistance to organic solvents and denaturing agents. Surprisingly, we have observed that DMSO and acetonitrile concentrations as high as 30%, and guanidinium concentrations as high as 1 M do not affect the stability of the capsid. The high stability of the capsid or VLP of Qβ is an advantageous feature, in particular, for its use in immunization and vaccination of mammals and humans in accordance of the present invention.

Further preferred virus-like particles of RNA-phages, in particular of Qβ and fr in accordance of this invention are disclosed in WO 02/056905, the disclosure of which is herewith incorporated by reference in its entirety. Particular example 18 of WO 02/056905 gave detailed description of preparation of VLP particles from Qβ.

In another preferred embodiment, the VLP of the invention is a VLP of RNA phage AP205. Assembly-competent mutant forms of AP205 VLPs, including AP205 coat protein with the substitution of proline at amino acid 5 to threonine, may also be used in the practice of the invention and leads to other preferred embodiments of the invention. WO 2004/007538 describes, in particular in Example 1 and Example 2, how to obtain VLP comprising AP205 coat proteins, and hereby in particular the expression and the purification thereto. WO 2004/007538 is incorporated herein by way of reference. AP205 VLPs are highly immunogenic, and can be linked with T-cadherin of the invention to typically and preferably generate vaccine constructs displaying the T-cadherin of the invention oriented in a repetitive manner. High antibody titer is elicited against the so displayed T-cadherin of the inventions showing that linked T-cadherin of the inventions are accessible for interacting with antibody molecules and are immunogenic.

In one preferred embodiment, the VLP of the invention comprises or consists of a mutant coat protein of a virus, preferably a RNA phage, wherein the mutant coat protein has been modified by removal of at least one lysine residue by way of substitution and/or by way of deletion. In another preferred embodiment, the VLP of the invention comprises or consists of a mutant coat protein of a virus, preferably a RNA phage, wherein the mutant coat protein has been modified by addition of at least one lysine residue by way of substitution and/or by way of insertion. In one very preferred embodiment, the mutant coat protein is of RNA phage Qβ, wherein at least one, or alternatively at least two, lysine residue have been removed by way of substitution or by way of deletion. In an alternative very preferred embodiment, the mutant coat protein is of RNA phage Qβ, wherein at least one, or alternatively at least two, lysine residue have been added by way of substitution or by way of insertion. In one further preferred embodiment, the mutant coat protein of RNA phage Qβ has an amino acid sequence selected from any one of SEQ ID NO:15-19. The deletion, substitution or addition of at least one lysine residue allows varying the degree of coupling, i.e. the amount of T-cadherin of the invention per subunits of the VLP of a virus, preferably of an RNA-phage, in particular, to match and tailor the requirements of the vaccine.

In one preferred embodiment, the compositions and vaccines of the invention have an antigen density being from 0.5 to 4.0. The term “antigen density”, as used herein, refers to the average number of T-cadherin of the invention which is linked per subunit, preferably per coat protein, of the VLP, and hereby preferably of the VLP of a RNA phage. Thus, this value is calculated as an average over all the subunits or monomers of the VLP, preferably of the VLP of the RNA-phage, in the composition or vaccines of the invention.

In another preferred embodiment of the present invention, the virus-like particle comprises, or alternatively consists essentially of, or alternatively consists of mutant coat protein of Qβ, or mutants or fragments thereof, and the corresponding A1 protein. In a further preferred embodiment, the virus-like particle comprises, or alternatively consists essentially of, or alternatively consists of mutant coat protein with amino acid sequence SEQ ID NO:15, 16, 17, 18, or 19 and the corresponding A1 protein.

In yet another preferred embodiment of the present invention, the virus-like particle comprises, or alternatively consists essentially of, or alternatively consists of a mixture of recombinant coat proteins, or fragments thereof, of the RNA-phage Qβ, AP205, fr or GA and of recombinant mutant coat proteins, or fragments thereof, of the RNA-phage Qβ, AP205, fr or GA.

Assembly-competent mutant forms of AP205 VLPs, including AP205 coat protein with the substitution of proline at amino acid 5 to threonine, asparigine at amino acid 14 to aspartic acid, may also be used in the practice of the invention and leads to other preferred embodiments of the invention. The cloning of the AP205Pro-5-Thr and the purification of the VLPs are disclosed in WO 2004/007538, and therein, in particular within Example 1 and Example 2. The disclosure of WO 2004/007538, and, in particular, Example 1 and Example 2 thereof is explicitly incorporated herein by way of reference.

Further RNA phage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Priano, C. et al., J. Mol. Biol. 249:283-297 (1995)). In particular the biological and biochemical properties of GA (Ni, C Z., et al., Protein Sci. 5:2485-2493 (1996), Tars, K et al., J. Mol. Biol. 271:759-773 (1997)) and of fr (Pusbko P. et al., Prot Eng. 6:883-891 (1993), Liljas, L et al. J. Mol. Biol. 244:279-290, (1994)) have been disclosed. The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. Another advantage of the VLPs derived from RNA phages is their high expression yield in bacteria that allows production of large quantities of material at affordable cost.

In one preferred embodiment, the at least one antigen is a T-cadherin domain protein. In a further preferred embodiment, the T-cadherin domain protein is a T-cadherin domain 1 protein, preferably of human origin.

In another preferred embodiment, the at least one antigen is a T-cadherin domain protein, wherein the T-cadherin domain protein comprises or consists of an amino acid sequence selected from the group consisting of: (a) SEQ ID NO: 40; (b) SEQ ID NO: 41; (c) SEQ ID NO: 42; (d) SEQ ID NO: 43; (e) SEQ ID NO: 44; and (f) an amino acid sequence which is at least 80%, or preferably at least 85%, more preferably at least 90%, or most preferably at least 95%, more preferably at least 97%, more preferably 99% identical with any one of SEQ ID NO: 40-44. Preferably the sequence difference of (f) to any one of (a) to (e) is due to deletion, insertion and substitution, further preferably due to conservative substitution. In an alternatively preferred embodiment, the T-cadherin domain protein comprises or consists of an amino acid sequence selected from the group consisting of: (a) SEQ ID NO: 24; (b) SEQ ID NO: 25; (c) SEQ ID NO: 26; (d) SEQ ID NO: 27; (c) SEQ ID NO: 28; and (f) an amino acid sequence which is at least 80%, preferably at least 85%, more preferably at least 90%, more preferably at least 95%, more preferably at least 97%, more preferably 99% identical with any one of SEQ ID NO: 24-28.

In one preferred embodiment, the at least one antigen is a combination of T-cadherin domain proteins. In a further preferred embodiment, the combination of T-cadherin domain proteins is a combination of T-cadherin domain 1 protein and T-cadherin domain 2 protein.

In one preferred embodiment, at least one antigen is a T-cadherin domain fragment, wherein said T-cadherin domain fragment comprises or alternatively consists of at least one antigenic site.

Methods to determine antigenic site(s) of a protein are known to the skilled person in the art. PCT/EP2005/004980, has elaborated some of these methods from the first paragraph of page 26 to the fourth paragraph of page 27 therein, and these specific disclosures are incorporated herein by reference. It is to be noted that these methods are generally applicable to other polypeptide antigens, and therefore are not restricted to IL-23 p19 as disclosed in PCT/EP2005/004980.

In one preferred embodiment of the invention, the VLP with at least one first attachment site is linked to the T-cadherin of the invention with at least one second attachment site via at least one peptide bond. Gene encoding of the invention, preferably T-cadherin domain fragment, more preferably a domain fragment not longer than 50 amino acids, even more preferably less than 30 amino acids, is in-frame ligated, either internally or preferably to the N- or the C-terminus to the gene encoding the coat protein of the VLP. Embodiments of fusing antigen of the invention to coat protein, mutants or fragments thereof, to a virus, preferably to an RNA phage, have been disclosed in WO 2004/009124 page 62 line 20 to page 68 line 17 and herein are incorporated by way of reference. The fusion protein shall preferably retain the ability of assembly into a VLP upon expression which can be examined by electromicroscopy.

In one preferred embodiment, a T-cadherin domain fragment is fused to either the N- or the C-terminus of a coat protein, mutants or fragments thereof, of RNA phage AP205. In one further preferred embodiment, the fusion protein further comprises a spacer, wherein said spacer is positioned between the coat protein, fragments or mutants thereof, of AP205 and a T-cadherin domain fragment.

In one preferred embodiment of the present invention, the composition comprises or alternatively consists essentially of a virus-like particle with at least one first attachment site linked to at least one T-cadherin of the invention with at least one second attachment site via at least one covalent bond, preferably the covalent bond is a non-peptide bond. In a preferred embodiment of the present invention, the first attachment site comprises, or preferably is, an amino group, preferably the amino group of a lysine residue. In another preferred embodiment of the present invention, the second attachment site comprises, or preferably is, a sulfhydryl group, preferably a sulfhydryl group of a cysteine. In another preferred embodiment of the present invention, the second attachment site comprises, or preferably is a maleimido group that that is associated, preferably, covalently associated with the at least one antigen.

In a very preferred embodiment of the invention, at least one first attachment site comprises or preferably is an amino group, preferably an amino group of a lysine residue and the at least one second attachment site comprises, or preferably is, a sulfhydryl group, preferably a sulfhydryl group of a cysteine.

In one preferred embodiment of the invention, the T-cadherin of the invention is linked to the VLP by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the VLP, and a further functional group which can react with the preferred second attachment site, i.e. a sulfhydryl group, preferably of cysteine(s) residue inherent of, or artificially added to the of the invention, and optionally also made available for reaction by reduction. Several hetero-bifunctional cross-linkers are known to the art. These include the preferred cross-linkers SMPH (Pierce), SulfoMBS, Sulfo-EMCS, Sulfo-GMBS, Sulfo-SIAB, Sulfo-SMPB, Sulfo-SMCC, SVSB, SIA and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. Another class of cross-linkers suitable in the practice of the invention is characterized by the introduction of a disulfide linkage between the T-cadherin of the invention and the VLP upon coupling. Preferred cross-linkers belonging to this class include, for example, SPDP and Sulfo-LC-SPDP (Pierce).

In a preferred embodiment, the composition of the invention further comprises a linker. Engineering of a second attachment site onto the T-cadherin of the invention is achieved by the association of a linker, preferably containing at least one amino acid suitable as second attachment site, according to the disclosures of this invention. Therefore, in a preferred embodiment of the present invention, a linker is associated to the T-cadherin of the invention by way of at least one covalent bond, preferably, by at least one, typically one peptide bond. Preferably, the linker comprises, or alternatively consists of, the second attachment site. In a further preferred embodiment, the linker comprises a sulfhydryl group, preferably of a cysteine residue. In another preferred embodiment, the amino acid linker is a cysteine residue.

The selection of a linker will be dependent on the nature of the T-cadherin of the invention, on its biochemical properties, such as pI, charge distribution and glycosylation. In general, flexible amino acid linkers are favored. In a further preferred embodiment of the present invention, the linker consists of amino acids, wherein further preferably the linker consists of at most 25, preferably at most 20, more preferably at most 15 amino acids. In an again preferred embodiment of the invention, the amino acid linker contains no more than 10 amino acids. Preferred embodiments of the linker are selected from the group consisting of: (a) CGG or CG/GC; (b) N-terminal gamma 1-linker (e.g. CGDKTHTSPP, SEQ ID NO:48); (c) N-terminal gamma 3-linker (e.g. CGGPKPSTPPGSSGGAP, SEQ ID NO: 59); (d) Ig hinge regions; (e) N-terminal glycine linkers (e.g. GCGGGG, SEQ ID NO:49); (f) (G)kC(G)n with n=0-12 and k=0-5; (g) N-terminal glycine-serine linkers ((GGGGS)n, n=1-3 with one further cysteine (for example SEQ ID NO:50, which corresponds to an embodiment wherein n=1); (h) (G)kC(G)m(S)l(GGGGS)n with n=0-3, k=0-5, m=0-10, l=0-2 (for example SEQ ID NO:51, which corresponds to an embodiment wherein n=1, k=1, l=1 and m=1); (i) GGC; (k) GGC-NH2; (l) C-terminal gamma 1-linker (e.g. DKTHTSPPCG, SEQ ID NO:52); (m) C-terminal gamma 3-linker (e.g. PKPSTPPGSSGGAPGGCG, SEQ ID NO:53); (n) C-terminal glycine linkers (GGGGCG, SEQ ID NO:54); (O) (G)nC(G)k with nOl2 and k=0-5; (p) C-terminal glycine-serine linkers ((SGGGG)n n=1-3 with one further cysteine (for example SEQ ID NO:55, which corresponds to an embodiment wherein n=1); (q) (G)m(S)l(GGGGS)n(G)oC(G)k with n=0-3, k=0-5, m=0-10, l=0-2, and o=0-8 (for example SEQ ID NO:56, which corresponds to an embodiment wherein n=1, k=1, l=1, o=1 and m=1). In a further preferred embodiment the linker is added to the N-terminus of T-cadherin of the invention. In another preferred embodiment of the invention, the linker is added to the C-terminus of T-cadherin of the invention.

Preferred linkers according to this invention are glycine linkers (G)n further containing a cysteine residue as second attachment site, such as N-terminal glycine linker (GCGGGG) and C-terminal glycine linker (GGGGCG). Further preferred embodiments are C-terminal glycine-lysine linker (GGKKGC, SEQ ID NO:57) and N-terminal glycine-lysine linker (CGKKGG, SEQ ID NO:58), GGCG a GGC or GGC-NH2 (“NH2” stands for amidation) linkers at the C-terminus of the peptide or CGG at its N-terminus. In general, glycine residues will be inserted between bulky amino acids and the cysteine to be used as second attachment site, to avoid potential steric hindrance of the bulkier amino acid in the coupling reaction.

The cysteine residue(s) served as the second attachment site, either inherent of or added to the T-cadherin of the invention, has to be in reduced state to react with the hetero-bifunctional cross-linker on the activated carrier, that is a free cysteine or a cysteine residue with a free sulfhydryl group has to be available.

Linking of the T-cadherin of the invention to the VLP by using a hetero-bifunctional cross-linker according to the preferred methods described above, allows coupling of the T-cadherin of the invention to the VLP in an oriented fashion. Other methods of linking the T-cadherin of the invention to the VLP include methods wherein the T-cadherin of the invention is cross-linked to the VLP, using the carbodiimide EDC, and NHS. The T-cadherin of the invention may also be first thiolated through reaction, for example with SATA, SATP or iminothiolane. The T-cadherin of the invention, after deprotection if required, may then be coupled to the VLP as follows. After separation of the excess thiolation reagent, the T-cadherin of the invention is reacted with the VLP, previously activated with a hetero-bifunctional cross-linker comprising a cysteine reactive moiety, and therefore displaying at least one or several functional groups reactive towards cysteine residues, to which the thiolated T-cadherin of the invention can react, such as described above. Optionally, low amounts of a reducing agent are included in the reaction mixture. In further methods, the T-cadherin of the invention is attached to the VLP, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the VLP.

In other embodiments of the present invention, the composition comprises or alternatively consists essentially of a virus-like particle linked to T-cadherin of the invention via chemical interactions, wherein at least one of these interactions is not a covalent bond. For example, linking of the VLP to the T-cadherin of the invention can be effected by biotinylating the VLP and expressing the T-cadherin of the invention as a streptavidin-fusion protein. Other binding pairs, such as ligand-receptor, antigen-antibody, can also be used as coupling reagent in a similar manner as biotin-avidin.

In very preferred embodiments of the invention, the T-cadherin of the invention is linked via a cysteine residue, having been added to either the N-terminus or the C-terminus of, or a natural cysteine residue within T-cadherin of the invention, to lysine residues of coat proteins of the VLPs of RNA phage, and in particular to the coat protein of Qβ.

U.S. Pat. No. 5,698,424 describes a modified coat protein of bacteriophage MS-2 capable of forming a capsid, wherein the coat protein is modified by an insertion of a cysteine residue into the N-terminal hairpin region, and by replacement of each of the cysteine residues located external to the N-terminal hairpin region by a non-cysteine amino acid residue. The inserted cysteine may then be linked directly to a desired molecular species to be presented such as an epitope or an antigenic protein.

We note, however, that the presence of an exposed free cysteine residue in the capsid may lead to oligomerization of capsids by way of disulfide bridge formation. Moreover, attachment between capsids and antigenic proteins by way of disulfide bonds are labile, in particular, to sulfhydryl-moiety containing molecules, and are, furthermore, less stable in serum than, for example, thioether attachments (Martin F J. and Papahadjopoulos D (1982) Irreversible Coupling of Immunoglobulin Fragments to Preformed Vesicles. J. Biol. Chem. 257: 286-288). Therefore, in a further very preferred embodiment, the linkage of the VLP and the at least one antigen does not comprise a disulfide bond. Further preferred hereby, the at least one second attachment comprise, or preferably is, a sulfhydryl group. Moreover, in again a very preferred embodiment of the invention, the linkage of the VLP and the at least one antigen does not comprise a sulphur-sulphur bond. In a further very preferred embodiment, said at least one first attachment site is not or does not comprise a sulfhydryl group of a cysteine. In again a further very preferred embodiment, said at least one first attachment site is not or does not comprise a sulfhydryl group.

In one preferred embodiment of the invention, the VLP is recombinantly produced in a host, and wherein the VLP is essentially free of host RNA, preferably host nucleic acids or wherein the VLP is essentially free of host DNA, preferably host nucleic acids. In one preferred embodiment, the VLP of an RNA phage is recombinantly produced in a host, and wherein the VLP of an RNA phage is essentially free of host RNA, preferably host nucleic acids.

In one further preferred embodiment, the composition further comprises at least one polyanionic macromolecule bound to, preferably packaged inside or enclosed in, the VLP. In a still further preferred embodiment, the polyanionic macromolecule is polyglutamic acid and/or polyaspartic acid. In one preferred embodiment, the VLP is of an RNA phage. Working embodiments as illustrative examples are provided in example 5 and 6 in this application.

Depending on the nature of the polyanionic macromolecule, the preferred molecular weight range varies. For example, for a polyanionic polypeptide, in particular for polyglutamic acids and polyaspartic acids, the preferred molecule weight is from 5000 Dalton to 150,000 Dalton. The lowest molecular weight is hereby preferably at least about 5000 Dalton, more preferably at least about 10,000 Dalton, even more preferably at least about 30,000 Dalton. The highest molecular weight is hereby preferably at most about 150,000 Dalton, preferably at most about 120,000 Dalton, even more preferably at most about 100,000 Dalton.

Reducing or eliminating the amount of host RNA, preferably host nucleic acids, minimizes or reduces unwanted T cell responses, such as inflammatory T cell responses and cytotoxic T cell responses, and other unwanted side effects, such as fever, while maintaining strong antibody response specifically against T-cadherin.

Essentially free of host RNA (or DNA), preferably host nucleic acids: The term “essentially free of host RNA (or DNA), preferably host nucleic acids” as used herein, refers to the amount of host RNA (or DNA), preferably host nucleic acids, comprised by the VLP, which is typically and preferably less than 30 μg, preferably less than 20 μg, more preferably less than 10 μg, even more preferably less than 8 μg, even more preferably less than 6 μg, even more preferably less than 4 μg, most preferably less than 2 μg, per mg of the VLP. Host, as used within the afore-mentioned context, refers to the host in which the VLP is recombinantly produced. Conventional methods of determining the amount of RNA (or DNA), preferably nucleic acids, are known to the skilled person in the art. The typical and preferred method to determine the amount of RNA, preferably nucleic acids, in accordance with the present invention is described in Example 17 of the PCT/EP2005/055009 filed on Oct. 5, 2005 by the same assignee. Identical, similar or analogous conditions are, typically and preferably, used for the determination of the amount of RNA (or DNA), preferably nucleic acids, for inventive compositions comprising VLPs other than Qβ. The modifications of the conditions eventually needed are within the knowledge of the skilled person in the art.

The term “polyanionic macromolecule”, as used herein, refers to a molecule of high relative molecular mass which comprises repetitive groups of negative charge, the structure of which essentially comprises the multiple repetitions of units derived, actually or conceptually, from molecules of low relative molecular mass.

In one aspect, the invention provides a vaccine composition comprising the composition of the invention. In one preferred embodiment, the T-cadherin of the invention linked to the VLP in the vaccine composition is of animal, preferably mammal or human origin.

In one preferred embodiment, the vaccine composition further comprises at least one adjuvant. The administration of at least one adjuvant may hereby occur prior to, contemporaneously or after the administration of the inventive composition. The term “adjuvant” as used herein refers to non-specific stimulators of the immune response or substances that allow generation of a depot in the host which when combined with the vaccine and pharmaceutical composition, respectively, of the present invention may provide for an even more enhanced immune response.

In another preferred embodiment, the vaccine composition is devoid of adjuvant. An advantageous feature of the present invention is the high immunogenicity of the composition, even in the absence of adjuvants. The absence of an adjuvant, furthermore, minimizes the occurrence of unwanted inflammatory T-cell responses representing a safety concern in the vaccination against self antigens. Thus, the administration of the vaccine of the invention to a patient will preferably occur without administering at least one adjuvant to the same patient prior to, contemporaneously or after the administration of the vaccine.

In a further aspect, the present invention provides for the use of the composition of the invention for the manufacture of a medicament for the treatment of T-cadherin-related diseases. In a preferred embodiment, the disease is selected from a group consisting of coronary artery disease, atherosclerosis, obesity, type I diabetes and type II diabetes and cancer.

The invention further discloses a method of immunization comprising administering the vaccine of the present invention to an animal or a human. The animal is preferably a mammal, such as cat, dog, rat and mouse. The vaccine may be administered to an animal or a human by various methods known in the art, but will normally be administered by injection, infusion, inhalation, oral administration, or other suitable physical methods. The conjugates may alternatively be administered intramuscularly, intravenously, transmucosally, transdermally, intranasally, intraperitoneally or subcutaneously. Components of conjugates for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

Vaccines of the invention are said to be “pharmacologically acceptable” if their administration can be tolerated by a recipient individual. Further, the vaccines of the invention will be administered in a “therapeutically effective amount” (i.e., an amount that produces a desired physiological effect). Without the intention to limit the present invention by the following mechanistic explanation, the inventive vaccine might induce antibodies which bind to T-cadherin and thus reducing its concentration and/or interfering with its physiological or pathological function.

In another aspect, the invention provides a pharmaceutical composition comprising the composition as taught in the present invention and an acceptable pharmaceutical carrier. When vaccine of the invention is administered to an individual, it may be in a form which contains salts, buffers, adjuvants, or other substances which are desirable for improving the efficacy of the conjugate. Examples of materials suitable for use in preparation of pharmaceutical compositions are provided in numerous sources including REMINGTON'S PHARMACEUTICAL SCIENCES (Osol, A, ed., Mack Publishing Co., (1990)).

In one further aspect, the invention provides a method of producing the composition of the invention, wherein the method comprises: (a) providing a VLP with at least one first attachment site; (b) providing at least one antigen, wherein said antigen is a T-cadherin of the invention selected from the group consisting of T-cadherin domain protein, a combination of T-cadherin domain proteins, a T-cadherin domain fragment and a combination of T-cadherin domain fragments, with at least one second attachment site; and (c) linking said VLP to said at least one antigen through said at least one first attachment site and said at least one second attachment site to produce said composition.

In a further preferred embodiment, the step of providing a VLP with at least one first attachment site further comprises steps: (a) disassembling said virus-like particle to said coat proteins, mutants or fragments thereof, of a virus; (b) purifying said coat proteins, mutants or fragments thereof; (c) reassembling said purified coat proteins, mutants or fragments thereof, to a virus-like particle, wherein said virus-like particle is essentially free of host RNA or free of host DNA, preferably free of host nucleic acids. In a still further preferred embodiment, the reassembling of said purified coat proteins, mutants or fragments thereof, is effected in the presence of at least one polyanionic macromolecule, preferably polyglutamic acid and/or polyaspartic acid. In one preferred embodiment, said VLP is a VLP of an RNA-bacteriophage.

In one aspect, the invention provides a method of treating a disease in an animal or a human comprising administering the inventive composition or the inventive vaccine of the invention to said animal or human, wherein said disease is selected form the group consisting of coronary artery disease, atherosclerosis, obesity, type I diabetes, type II diabetes and cancer, preferably in cancer in which angiogenesis plays an important role. In one preferred embodiment, the invention provides a method of preventing or treating angiogenesis in a cancer.

In one aspect, the invention provides a substance, wherein said substance is characterized by being capable of binding to T-cadherin and wherein said substance modulates the function of T-cadherin, and wherein preferably modulates the function of T-cadherin in the diseases selected from the group consisting of: coronary artery disease, atherosclerosis, obesity, type I diabetes, type II diabetes and cancer, preferably in cancer in which angiogenesis plays an important role.

In one aspect, this invention provides a substance, wherein said substance is an antibody, preferably a monoclonal antibody, wherein said antibody specifically binds to T-cadherin, preferably specifically binds to T-cadherin domain 1.

In one preferred embodiment, said antibody is produced in response to the composition of the invention, i.e. a composition comprising VLP-T-cadherin according to the invention. In one further preferred embodiment, said antibody is produced in response to VLP-T-cadherin domain 1, further preferably in response to Qβ-T-cadherin domain 1. An antibody in response to the composition of the invention is typically and preferably produced by immunizing animal or human, preferably mouse, according to routine practice in the art.

In one preferred embodiment, said antibody is a polyclonal antibody. In one preferred embodiment, said antibody is a monoclonal antibody. Monoclonal antibody, depending on the techniques used, may be a murine, a chimeric, a CDR-grafted, a humanized, a human or a synthesized antibody. Thus the term “monoclonal antibody” means an antibody composition having a homogeneous antibody population. It is not intended to be limited as regards to the source of the antibody or the manner in which it is made. In one preferred embodiment, said substance comprises or is a functional fragment of said antibody.

In one aspect, the invention provides a method of treating a disease in an animal or a human comprising administering at least one substance to said animal or human, wherein said substance is characterized by being capable of binding to T-cadherin and wherein said disease is selected form the group consisting of coronary artery disease, atherosclerosis, obesity, type I diabetes, type II diabetes and cancer, preferably in cancer in which angiogenesis plays an important role. In one aspect, the invention provides a use of at least one substance for the manufacture of a medicament for the treatment of a disease in an animal or a human, wherein said substance is characterized by being capable of binding to T-cadherin and wherein said disease is selected form the group consisting of coronary artery disease, atherosclerosis, obesity, type I diabetes, type II diabetes and cancer, preferably in cancer in which angiogenesis plays an important role.

The phrase “a substance is characterized by being capable of binding to T-cadherin”, as used herein, means that the dissociation constant of T-cadherin and said substance is higher than 1×10⁻⁶ preferably higher than 10⁻⁷, preferably higher than 10⁻⁸, further preferably higher than 10⁻⁹. Methods to determine the dissociation constant of T-cadherin and said substance is known to a skilled artisan (Current Protocol in Protein Science, Vol 1: 3.5.8 and reference cited therein).

Substances that are capable of binding to T-cadherin can be identified by various methods. Typically and preferably small chemical compound, peptide or antibody libraries are used in a screening assay, preferably in vitro, using T-cadherin as a bait. Further preferably, the assay is a high through put assay. Methods of screening compound that bind to a receptor, preferably high through put screening assays, are known to skilled artisans.

In one preferred embodiment, the invention provides a method of treating atherosclerosis in an animal, preferably a dog or a cat, preferably a domestic cat, or a human comprising administering at least one substance to said animal or human.

In another preferred embodiment, the invention provides a method of treating cancer in an animal, preferably a dog or a cat, preferably a domestic cat, or a human comprising administering said at least one substance to said animal or human. In one further preferred embodiment, the invention provides a method of preventing or treating angiogenesis in a cancer comprising administering said at least one substance to said animal or human.

In one preferred embodiment, the at least one substance is an antibody.

In one preferred embodiment, the at least one substance are polyclonal antibodies. In one preferred embodiment, the polyclonal antibodies are isolated from a human or a animal, preferably a mouse, immunized with VLP-T-cadherin of the invention, further preferably immunized with VLP-T-cadherin domain 1, still further preferably immunized with Qβ-T-cadherin domain 1. In one preferred embodiment, T-cadherin domain 1 is used as antigen to generate the polyclonal antibodies. In one further preferred embodiment, VLP-T-cadherin domain 1 is used as antigen, even more preferably Qβ-T-cadherin domain 1 is used as antigen.

Other polyclonal antibodies directed against the T-cadherin extracellular domains or peptides thereof have been described in the art. For example polyclonal antibodies against the human extracellular domain 1 have been described in Ivanov et al., Histochem. Cell. Biol. 115:231-242 (2001); polyclonal antibodies against the human extracellular domain 1 to 5 have been described in Fredette et al Development 122, 3163-3171 (1996); antibodies against various T cadherin peptides have been described in Sidorova er al. Bioorg Khim 25(3) 171-178 (1999), Sacristan et al J. Neurosci. Res. 34, 664-680 (1993), Philippova et al. FEBS Letters 429 207-210 (1998), Zhong et al. Clin Cancer Res. (6):1683-7 (2001), Lee S W Nat. Med. 1996 July; 2(7):776-82, Santa Cruz Biotechnology INC(H126: No sc6457, C19: No. 6460).

Once the polyclonal antibodies are proved to be useful in the disease models of coronary artery disease, atherosclerosis, obesity, type I diabetes, type II diabetes and cancer, preferably in cancer in which angiogenesis plays an important role, the polyclonal antibodies can be further purified. Preferably the same antigen used to generate the useful polyclonal antibodies will be used again in the generation of monoclonal antibodies. The generated monoclonal antibodies will be further evaluated for their efficacy in these disease models.

In one preferred embodiment, the at least one substance is a monoclonal antibody. Methods of raising polyclonal and monoclonal antibodies that are specifically for T-cadherin are known to the skilled person in the art. For example, to generate monoclonal antibodies that recognize T-cadherin or a fragment thereof, animals, preferably mice or rats, and more preferably mice with a humanized B cell repertoire, can be injected with T-cadherin or a fragment of T-cadherin. Alternatively, the animal may be injected with DNA encoding T-cadherin or a fragment thereof. The DNA molecule is preferably linked to a T helper cell epitope. Monoclonal antibodies are generated thereafter using standard methods (see e.g. Chapter 6, Antibodies A Laboratory Manual, Cold Spring Harbor Laboratory Press, 1988).

In one preferred embodiment, the monoclonal antibody specifically binds to T-cadherin, preferably human T-cadherin. In one preferred embodiment, the monoclonal antibody specifically binds to any one of the five extracellular domains of T-cadherin, preferably human T-cadherin. In one further preferred embodiment, the monoclonal antibody specifically binds to extracellular domain 1 of T-cadherin, preferably human T-cadherin domain 1.

In one preferred embodiment, the at least one substance is a monoclonal antibody produced by immunizing mouse with T-cadherin domain 1, preferably with VLP-T-cadherin domain 1 of the invention, still further preferably with Qβ-T-cadherin domain 1 of the invention. In one preferred embodiment, the at least one substance is a monoclonal antibody generated by immunizing mouse with at least one T-cadherin domain 1 fragment, either alone or couples to a carrier, wherein said carrier is preferably a VLP, further preferably an RNA phage, further preferably Qβ.

In one preferred embodiment, the at least one substance comprises or is a protein or a peptide. In one preferred embodiment, the at least one substance comprises or is a soluble T-cadherin. In one preferred embodiment, the at least one substance comprises or is any one of the five T-cadherin extracellular domain protein or any combination thereof. In one further preferred embodiment, the at least one substance comprises or is any one of the five T-cadherin extracellular domain or any combination thereof. In one preferred embodiment, the at least one substance comprises or is a T-cadherin domain 1 protein, preferably T-cadherin domain 1. In one preferred embodiment, the at least one substance comprises or is a combination of T-cadherin domain 1 protein, preferably T-cadherin domain 1, and any other extracellular domain protein, preferably any other extracellular domain, wherein preferably said any other extracellular domain protein is T-cadherin domain 2 protein, preferably T-cadherin domain 2.

In one preferred embodiment, the at least one substance comprises or is a peptide. In one preferred embodiment, said peptide is a T-cadherin domain 1 fragment. In one further preferred embodiment, said T-cadherin domain 1 fragment derived from homologous regions to the ones that have been shown to modulate N-Cadherin homophilic interactions (Williams et al. Mol Cell Neurosci, 2000. 15(5): p. 456-64), Williams et al, J. Biol. Chem. 2002, 277 (6), p4361-4367.) In one preferred embodiment, said peptide comprises or consisting of an amino acid sequence selected from the group consisting of: (a) GVD; (b) INENTGS (SEQ ID NO:60); (c) ETITDV (SEQ ID NO:61); (d) any combination of (a) to (c); and (e) any repetition of (a) to (d), preferably at least two times, more preferably two times repetition. In one preferred embodiment, said peptide further comprises spacers positioned in between elements comprising (a), (b) or (c).

In one preferred embodiment, said peptide is a cyclic peptide. Preferably said peptide is cyclized through 2 flanking cysteins, the N-terminus of the sequences is acetylated and the C-terminus is amidated. In one preferred embodiment, the cyclic peptide comprises or consists of an amino acid sequence selected from the group consisting of: (a) N—Ac—C—INENTGS—C—NH2 or tandem repeats thereof; (b) N—Ac—C-ETTDVNGETIDV-C—NH2; and (c) N—Ac—C—INENTGSINENTGS—C—NH2. Methods for the generation of cyclic peptides are well known to skilled artisans and have been previously described in the art (Williams et al. Mol Cell Neurosci, 2000. 15(5): p. 456-64). In one preferred embodiment, the at least one substance is an antibody produced in response to the peptide as defined herein.

In one preferred embodiment, the at least one substance is a substance that does not naturally exist in vivo or does not exhibit physiological function in vivo. Preferably said substance is not adiponectin.

In one preferred embodiment, the at least one substance is a substance that does not interfere with the binding between T-cadherin and Adiponectin. The phrase “a substance does not interfere with the binding between T-cadherin and Adiponectin”, as used herein, refers to a substance, which when tested in a T-cadherin and adiponectin binding experiment, does not reduce the binding between T-cadherin and adiponectin by more than 50%, preferably 30%, more preferably 20%, even more preferably 10%, even more preferably 5%, compared to the substance used as a negative control. The substance used as negative control is tested at the same concentration and in the same experiment as the substance to be tested. “The same experiment”, as used herein, refers to that the substance to be tested and the substance used as a negative control are tested in parallel and in an identical setting of one experiment. “The same concentration”, as used herein, refers to that the concentration, which can be expressed by gram/liter, by molar, etc, of the substance to be tested and the substance as a negative control as not different from each other by more than 5%, preferably by more than 2%.

A substance used as a negative control should have similar molecular weight as the substance to be tested (preferably not different by more than 20% from each other) or have similar structure as the substance to be tested. For example, if a serum from a immunized animal is the substance to be tested, then preimmune serum from the same animal or serum from an animal immunized with another unrelated antigen should be used as a negative control. If a monoclonal antibody is the substance to be tested, then another monoclonal antibody, which is irrelevant from the components in the experiment, should be used as a negative control. If a peptide is the substance to be tested, then another peptide with irrelevant sequence from the components in the experiment, should be used as a negative control.

A typical and preferred experiment to test whether a substance interfere with T-cadherin and Adiponectin binding comprising: (a) obtaining a cell line that over expresses T-cadherin; (b) adding adiponectin to the cell culture; (c) prior to, contemporaneously or after the addition of adiponectin, add the substance to be tested to the same cell culture. Add the substance as a negative control to a parallel cell culture with identical setting (d) preferably the substance to be tested and the substance used as a negative control are tested in a series of dilutions; (e) determine the binding of adiponectin to T-cadherin in the presence of the substance to be tested or the substance as a negative control. Preferably in step (e), the binding of adiponectin to T-cadherin is determined and quantified by fluorescence activated cell sorter (FACS) analysis. Briefly, the bound adiponectin is stained with fluorescently labeled anti-adiponectin antibody or with first antibody against adiponectin and fluorescently labeled secondary antibody against the first antibody. The amount of bound adiponectin can than be quantified through the measurement of the geometric mean fluorescence of the cells. Similarly and well known to a skilled person, ELISA can also be used to test whether a substance interfere with T-cadherin and adiponectin binding.

In one aspect, the invention provides a substance, wherein said substance is an anti-sense RNA or siRNA targeting T-cadherin. In one aspect, the invention provides a method of treating a disease comprising the administration of the anti-sense RNA or siRNA targeting T-cadherin to an animal, preferably to a mouse, cat or dog or to a human, wherein said disease is selected from the group consisting of: coronary artery disease, atherosclerosis, obesity, type I diabetes, type II diabetes and cancer, preferably in cancer in which angiogenesis plays an important role. The administering of anti-sense RNA or siRNA targeting T-cadherin will lower the expression of T-cadherin. Without being bound by the theory, the reduction of T-cadherin may improve the pathological condition of the aforementioned diseases, in particular in coronary artery disease and in atherosclerosis.

### EXAMPLES

The terms “prior art VLPs” as well as the more specific terms “prior art Qβ VLPs”, “prior art AP205 VLPs” and the like, as used within this example section, refer to VLPs obtained by recombinant expression from E. coli and subsequent purification as described in WO 02/056905, WO 04/007538.

### Example 1

### Cloning of the T-Cadherin CAD1

A cDNA library derived from differentiated mouse C2C12 cells was used as a template for PCR amplification of CAD1 (SEQ ID NO:24), using the primer pair Fwd CAD-3: CTAGCTAGCTCCATTGTGGTGTCCCCCA (SEQ ID NO: 29) Rev-CAD-6: CTACTCGAGGAAGATGGGTCTGTTGTCG (SEQ ID NO: 30). The forward primer contains a NheI site and the reverse primer an XhoI site allowing the cloning of CAD1 into plasmid pMOD-EC3 (as described in EXAMPLE 4 of US 2003-0175290-A1). The PCR fragment was cloned into pMOD-EC3 and clones were sequenced, the resulting plasmid was named pMOD-C6xCAD1.

### Example 2

### Expression and Purification of His-Tagged CAD1

The plasmid pMOD-C6xCAD1 was transformed into the bacterial expression strain BL21 (DE3) (Novagen). The expression was induced at OD600 of 1.0 by adding IPTG to a final concentration of 1 mM. The culture was grown for an additional 3 hours and the cells harvested by centrifugation. The cells were resuspended in 10 ml ice-cold native lysis buffer (50 mM NaH₂PO₄, 300 mM NaCl, 10 mM imidazole pH 8.0) and disrupted by sonication.

The clarified bacterial lysate was brought to 50 ml with native lysis buffer: One ml of nickel-nitrilotriacetic acid (Ni-NTA) agarose (Qiagen) was added to the lysate and the lysate was further incubated by inverting for 1 hour at 4° C. allowed binding of the His-tagged CAD1 fusion protein to the agarose.

The collected agarose was washed by lysis buffer for 4 times. Bound protein was eluted by resuspension of the Ni-NTA agarose in 2 ml of elution buffer (50 mM NaH₂PO₄, 300 mM NaCl, 250 mM imidazole pH 8.0). Elution of the protein was repeated 2-3 times. Elution buffer was exchanged for Dulbecco's PBS (Gibco) using a Amicon Ultra-15 centrifugal filter device (Millipore). Aliquots of CAD1 were snap-frozen in liquid nitrogen and stored overnight at −80° C.

### Example 3

### Coupling by Chemical Cross-Linking of CAD1 to Prior Art Qβ VLPs

A solution of 143 μM prior art Qβ VLP in HEPES buffer (20 mM HEPES, 150 mM NaCl, pH 7.2) was reacted with a 5-fold molar excess (715 μM) of SMPH (Pierce) for 30 minutes at 25° C. with shaking. Reaction products were dialyzed against two changes of Dulbecco's PBS (Gibco) using a dialysis unit with a 10,000 Da molecular weight cutoff (Slide-A-Lyzer, Pierce).

Stored CAD1 aliquots and SMPH-derivatized Qβ VLPs were thawed to room temperature. Before coupling, CAD1 was incubated with TCEP (Pierce, Perbio Science) in equimolar amounts for 30 minutes at room temperature. Subsquently, CAD1 was added in a 5-fold molar excess to a 143 μM SMPH-derivatized Qβ VLPs. Reaction volume was 650 μl and multiple reactions were performed in parallel. Reactions were incubated for 4 hours at room temperature with shaking. After coupling, aliquots were centrifuged at 16,000×g for 3 minutes at 4° C. to pellet insoluble material. The supernatants were pooled in fresh tubes.

The coupling of CAD1 to Qβ VLP was assessed by reducing SDS-PAGE. Coupling efficiency was approximately 50%.

### Example 4

### Cloning, Expression and Purification of Other CAD Domains

Other CAD domains were cloned into the expression vector pMOD-EC3. The sequences of primers used in the cloning are summarized in TABLE 1.

| CAD domains | Primer Sequences | The Resulting Plasmids |
|---|---|---|
| CAD2 (SEQ ID NO: 25) | SEQ ID NO: 31 | pMOD-C6xCAD2 |
| | SEQ ID NO: 32 | |
| CAD3 (SEQ ID NO: 26) | SEQ ID NO: 33 | pMOD-C6xCAD3 |
| | SEQ ID NO: 34 | |
| CAD4 (SEQ ID NO: 27) | SEQ ID NO: 3511 | pMOD-C6xCAD4 |
| | SEQ ID NO: 36 | |
| CAD5 (SEQ ID NO: 28) | SEQ ID NO: 37 | pMOD-C6xCAD5 |
| | SEQ ID NO: 38 | |

The expression and purification of the above mentioned proteins are carried out substantially the same as described in EXAMPLE 2.

### Example 5

### Preparation of Qβ VLps of the Invention by Disassembly/Reassembly in the Presence of Different Polyanionic Macromolecules Resulting in Reassembled Qβ VLPs

### (A) Disassembly of Prior Art Qβ VLP

45 mg prior art Qβ VLP (2.5 mg/ml, as determined by Bradford analysis) in PBS (20 mM Phosphate, 150 mM NaCl, pH 7.5) purified from E. coli lysate was reduced with 10 mM DTT for 15 min at room temperature under stirring conditions. Magnesium chloride was then added to 0.7 M final concentration and the incubation was continued for 15 min at room temperature under stirring conditions, which led to the precipitation of the encapsulated host cell RNA. The solution was centrifuged for 10 min at 4000 rpm at 4° C. (Eppendorf 5810 R, in fixed angle rotor A-4-62 used in all following steps) in order to remove the precipitated RNA from the solution. The supernatant, containing the released, dimeric Qβ coat protein, was used for the chromatographic purification steps.

### (B) Purification of the Qβ Coat Protein by Cation Exchange Chromatography and by Size Exclusion Chromatography

The supernatant of the disassembly reaction, containing the dimcric coat protein, host cell proteins and residual host cell RNA, was diluted 1:15 in water to adjust conductivity below 10 mS/cm and was loaded onto a SP-Sepharose FF column (xk16/20, 6 ml, Amersham Bioscience). The column was equilibrated beforehand with 20 mM sodium phosphate buffer pH 7. The elution of the bound coat protein was accomplished by a step gradient to 20 mM sodium phosphate/500 mM sodium chloride and the protein was collected in a fraction volume of approx. 25 ml. The chromatography was carried out at room temperature with a flow rate of 5 ml/min and the absorbance was monitored at 260 nm and 280 mm.

In the second step, the isolated Qβ coat protein (the eluted fraction from the cation exchange column) was loaded (in two runs) onto a Sephacryl S-100 HR column (xk26/60, 320 ml, Amersham Bioscience), equilibrated with 20 mM sodium phosphate/250 mM sodium chloride; pH 6.5. The chromatography was carried out at room temperature with a flow rate of 2.5 ml/min and the absorbance was monitored at 260 nm and 280 nm. Fractions of 5 ml were collected.

### (C1) Reassembly of the Qβ VLP by Dialysis

Purified Qβ coat protein (2.2 mg/ml in 20 mM sodium phosphate pH 6.5), one polyanionic macromolecule (2 mg/ml in water), urea (7.2 M in water) and DTT (0.5 M in water) were mixed to the final concentrations of 1.4 mg/ml coat protein, 0.14 mg/ml of the respective polyanionic macromolecule, 1 M urea and 2.5 mM DTT. The mixtures (1 ml each) were dialyzed for 2 days at 5° C. in 20 mM Tris HCl, 150 mM NaCl pH 8, using membranes with 3.5 kDa cut off. The polyanionic macromolecules were: polygalacturonic acid (25000-50000, Fluka), dextran sulfate (MW 5000 and 10000, Sigma), poly-L-aspartic acid (MW 11000 and 33400, Sigma), poly-L-glutamic acid (MW 3000, 13600 and 84600, Sigma) and tRNAs from bakers yeast and wheat germ.

### (C2) Reassembly of the Qβ VLP by Diafiltration

33 ml purified Qβ coat protein (1.5 mg/ml in 20 mM sodium phosphate pH 6.5, 250 mM NaCl) was mixed with water and urea (7.2 M in water), NaCl (5 M in water) and poly-L-glutamic acid (2 mg/ml in water, MW: 84600). The volume of the mixture was 50 ml and the final concentrations of the components were 1 mg/ml coat protein, 300 mm NaCl, 1.0 M urea and 0.2 mg/ml poly-L-glutamic acid. The mixture was then diafiltrated at room temperature, against 500 ml of 20 mM Tris HCl pH 8, 50 mM NaCl, applying a cross flow rate of 10 ml/min and a permeate flow rate of 2.5 ml/min, in a tangential flow filtration apparatus using a Pellicon XL membrane cartridge (Biiomax 5K, Millipore).

### Example 6

### In Vitro Assembly of AP205 VLPs

### (A) Purification of AP205 Coat Protein

Disassembly: 20 ml of AP205 VLP solution (in 1.6 mg/ml PBS, purified from E. coli extract) was mixed with 0.2 ml of 0.5 M DTT and incubated for 30 min at room temperature. 5 ml of 5 M NaCl was added and the mixture was then incubated for 15 min at 60° C., causing precipitation of the DTT-reduced coat proteins. The turbid mixture was centrifuged (rotor Sorvall SS34, 10000 g, 10 min, 20° C.) and the supernatant was discarded and the pellet was dispersed in 20 ml of 1 M Urea/20 mM Na Citrate pH 3.2. After stirring for 30 min at room temperature, the dispersion was adjusted to pH 6.5 by addition of 1.5 M Na₂HPO₄ and then centrifuged (rotor Sorvall SS34, 10000 g, 10 min, 20° C.) to obtain supernatant containing dimeric coat protein.

Cation exchange chromatography: The supernatant (see above) was diluted with 20 ml water to adjust a conductivity of approx. 5 mS/cm. The resulting solution was loaded on a column of 6 ml SP Sepharose FF (Amersham Bioscience) which was previously equilibrated with 20 mM sodium phosphate pH 6.5 buffer. After loading, the column was washed with 48 ml of 20 mM sodium phosphate pH 6.5 buffer followed by elution of the bound coat protein by a linear gradient to 1 M NaCl over 20 column volumes. The fractions of the main peak were pooled and analyzed by SDS-PAGE and UV spectroscopy. According to SDS-PAGE, the isolated coat protein was essentially pure from other protein contaminations. According to the UV spectroscopy, the protein concentration was 0.6 mg/ml (total amount 12 mg), taking that 1 A280 unit reflects 1.01 mg/ml of AP205 coat protein. Furthermore, the value of A280 (0.5999) over the value of A260 (0.291) is 2, indicating that the preparation is essentially free of nucleic acids.

### (B) Assembly of AP205 VLPs

Assembly in the absence of any polyanionic macromolecule: The eluted protein fraction from above was diafiltrated and concentrated by TFF to a protein concentration of 1 mg/ml in 20 mM sodium phosphate pH 6.5. 500 μl of that solution was mixed with 50 μl of 5 M NaCl solution and incubated for 48 h at room temperature. The formation of reassembled VLPs in the mixture was shown by non-reducing SDS-PAGE and by size exclusion HPLC. A TSKgel G5000 PWXL column (Tosoh Bioscience), equilibrated with 20 mM sodium phosphate, 150 mM NaCl pH 7.2, was used for the HPLC analysis.

Assembly in the presence of polyglutamic acid: 375 μl of purified AP205 coat protein (1 mg/ml in 20 mM sodium phosphate pH 6.5) was mixed with 50 μl of NaCl stock solution (5 M in water) solution, 50 μl of polyglutamic acid stock solution (2 mg/ml in water, MW: 86400, Sigma) and 25 μl of water. The mixture was incubated for 48 h at room temperature. The formation of reassembled VLP in the mixture was shown by non-reducing SDS-PAGE and by size exclusion HPLC. The coat protein in the mixture was almost completely incorporated into the VLPs, showing higher assembly efficiency than the AP205 coat protein assembled in the absence of any polyanionic macromolecule.

### Example 7

### Coupling T-Cadherin CAD Domains to the Reassembled VLPs

A solution of 143 μM reassembled Qβ VLP obtained from EXAMPLE 5 or AP205 VLP obtained from EXAMPLE 6 in HEPES buffer (20 mM HEPES, 150 mM NaCl, pH 7.2) are derivatized essentially as described in EXAMPLE 3.

Purified T-cadherin domains CAD1, CAD 2, CAD3, CAD4 or CAD 5 protein is incubated incubated with TCEP (Pierce, Perbio Science) in equimolar amounts for 30 minutes at room temperature. Subsquently, CAD domain is added in a 5-fold molar excess to a 143 μM SMPH-derivatized reassembled Qβ VLP or AP205 VLP. Reaction volume is 650 μl and multiple reactions are performed in parallel. Reactions are incubated for 4 hours at room temperature with shaking. After coupling, aliquots are centrifuged at 16,000×g for 3 minutes at 4° C. to pellet insoluble material. The supernatants containing the coupled conjugates are pooled in fresh tubes.

### Example 8

### Immunization of Apoe^{-/-} Mice Fed a Western Diet with Qβ VLP coupled to CAD1 and Analysis of Atherosclerosis

Seven to eight weeks old male Apoe^{-/-} mice (The Jackson Laboratory, Bar Harbor Me.) were injected subcutaneously with either 50 μg prior art Qβ VLP coupled to CAD1 (obtained from EXAMPLE 3) (n=6) or with 50 μg prior art Qβ VLP only (n=4) on day 0, 14, 28, 56 and 102. The mice were fed initially a normal chow diet, which was replaced on day 21 by a western diet (20% fat, 0.15% cholesterol). The mice were bled on day 0, 14, 23, 56 and 102, and the antibody response against T-cad1 was measured in the sera. They were sacrificed on day 159, and the aorta was isolated and prepared essentially as described (Tangirala R. K. et al. (1995) J. Lipd. Res. 36: 2320-2328). Briefly, the mice were bled by cardiac puncture and perfused with cold PBS. The aorta was then exposed, as much of the adventitia removed in situ, and the aorta finally removed from the heart. The aorta was further cleaned from residual adventitia on a glass petri dish filled with cold PBS, and the arch of the aorta was sectioned 5 mm down from the left sub clavian artery. The aorta were cut longitudinally, pinned out on a black wax surface and fixed overnight in 4% formalin. They were then stained overnight in oil red O. The plaques were quantified with an imaging software (Motic Image Plus 2.0) on digital photographs. The plaque load was expressed as the sum of the surface of all plaques of the aorta taken up to the iliac bifurcation, divided by the total surface of the aorta measured up to the iliac bifurcation. The difference in median of the plaque load between the Qβ-Tcad1 and Qβ group was analysed with a Mann-Whitney test, using a two-tailed p-value.

The antibody response was measured by ELISA, by coating the recombinant T-cadherin domain 1 protein (CAD1)(expressed and purified as described in EXAMPLE 2) to 96-well plates (Nunc Immuno MaxiSorp) Binding of specific antibodies was detected using a goat anti-mouse HRP conjugate. The titers against Tcad1 on day 14, 23, 56 and 102 were 2747±1817, 25134±16432, 15536±5895 and 7251±4368, respectively. No response was detected in pre-immune sera. A sustained and high-titer antibody response was thus raised against Tcad1, demonstrating that vaccination with Tcad1 coupled to the VLP Qβ was able to break self-tolerance and induce a high antibody titer against the self-antigen T-cadherin.

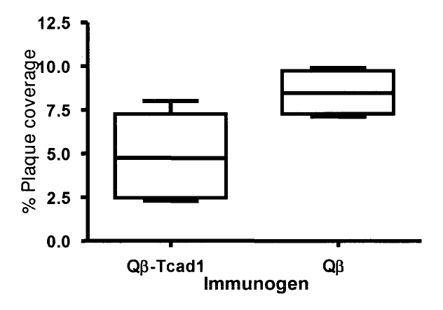

In addition, vaccination against Tcad1 significantly decreased median plaque load to 4.8% in the Qβ-Tcad1 group, as compared to 8.5% in the Qβ only vaccinated group (p=0.04; FIG. 2). Statistical analysis was performed with a Mann Whitney test, with a two-tailed p-value. Thus, surprisingly, vaccination against Tcad1 decreased atherosclerosis in Apoe^{-/-} mice.

The extent of atherosclerosis in each animal is further evaluated by histological analysis of cross-sections through the aortic origin, as described by Ludewig B. et al. (PNAS, (2000) 97:12752-12757). Frozen serial cross-sections through the aortic origin are harvested beginning with the appearance of all three valve cusps. They are stained with oil red 0 and counter stained with hematoxylin to quantify lesion size.

### Example 9

### Immunization of Mice with Qβ-Tcad1

C57BL/6 mice are vaccinated with the prior art Qβ-Tcad1 or the reassembled Qβ-Tcad1 obtained in EXAMPLE 3 and EXAMPLE 7 respectively. Briefly, 100 μg of dialyzed vaccine from each sample is diluted in PBS to a volume of 200 μl and injected subcutaneously (100 μl on each of the two ventral sides) on days 0, 14, 28 and 42 and subsequently as required. The vaccine is administered with or without adjuvant. As a control, a group of mice is immunized with the prior art Qβ VLP, the reassembled Qβ VLP, or injected with PBS, with or without adjuvant. Mice are bled retro-orbitally on day 0, 14, 28, 42 and subsequently at regular intervals. The T-cadherin specific antibodies are then quantified by ELISA as described in EXAMPLE 8.

### Example 10

### The Effect of Qβ-Tcad1 on the Development of Obesity

Adult, male or female, C57BL/6 mice with comparable starting weights are vaccinated, as described in EXAMPLE 9. Mice are subsequently boosted if T cadherin-specific antibody titers significantly decline during the experiment. All mice are placed on a high fat diet (35% fat by weight, 60% as energy) to facilitate the development of diet-induced obesity. Food and water is administered ad libitum. Body weights are monitored at regular intervals. Blood glucose levels are determined using the Glucotrend blood glucose meter (Roche) according to the manufacturer's recommendation. Triglyceride levels are measured after a 12 h fasting period. Briefly, blood samples are taken and triglyceride levels are determined from plasma samples by enzymatic assays with an Olympus AU400 automated laboratory work station. Body fat mass is measured by dual energy X-ray absorptiometry scan (DEXA). DEXA analyses are performed by the ultra high resolution PIXIMUS Series Densitometer (0.18×0.18 mm pixels, GE Medical Systems).

### Example 11

The effect of Qβ-Tcad1 on Obesity and Blood Glucose Levels in a Diet-Induced Animal Model of Obesity

Adult Male of female, C57BL/6 mice are fed a high fat diet, ad libitum, for approximately 17 weeks until they have become obese (weights>45 g). The mice are then vaccinated, as described in EXAMPLE 9. Mice are subsequently boosted if T cadherin-specific antibody titers significantly decline during the experiment. Body weights are monitored at regular intervals. In addition body fat mass, blood glucose levels and plasma triglyceride levels are determined at different intervals, as described in EXAMPLE 10.

### Example 12

### The Effect of Qβ-Tcad1 on Blood Glucose Levels in a Mouse Model for type I Diabetes

As a genetic model for type I diabetes, none obese diabetic mice (NOD) which spontaneously develop diabetes are used. These mice have low but still measurable levels of insulin after the onset of diabetes. Adult male or female NOD mice are vaccinated, essentially the same as described in EXAMPLE 9. Mice are subsequently boosted if T cadherin-specific antibody titers significantly decline during the experiment Mice are fed a standard diet (consisting of 4-10% fat by weight), ad libitum, and have free access to water. Mice are bled at regular intervals and blood glucose levels are determined as described in EXAMPLE 10.

### Example 13

### The Effect of Qβ-Tcad1 on Insulin Resistance, Glucose Tolerance and Insulin Secretion in Obese Mice

Adult Male of female, C57BL/6 mice are fed a high fat diet, ad libitum, for approximately 17 weeks until they have become obese (weights>45 g). The mice are then vaccinated, as described in EXAMPLE 9. Mice are subsequently boosted if T cadherin-specific antibody titers significantly decline during the experiment.

At different intervals, typically every week the insulin responsiveness of the mice is determined. Briefly, in the insulin tolerance test (ITT) the mice are injected with 0.5 U/kg human insulin (Novonordisk) subcutaneously in the fed condition. Blood samples are then taken from the tail vein every 20 minutes from immediately before the injection up to 2 h after the injection and the blood glucose levels are determined as described in EXAMPLE 10.

At different intervals, typically every one to two weeks glucose tolerance (GTT) and glucose induced insulin secretion is determined. Briefly, after a 16 h fasting of the animals, D-glucose (2 g/kg body weight) is loaded orally. Blood samples are collected immediately before and 10, 20, 30, 60 90 and 120 minutes after glucose load through the tail vain. The blood glucose levels are then determined as described in EXAMPLE 10 and the insulin levels are determined by ELISA.

### Example 14

### Identification of Antibodies that Recognize T Cadherin

A) Generation of T Cadherin Specific Antibodies

A C57BL/6 mouse was immunised with 50 μg prior art Qβ-Tcad1 vaccine (obtained in EXAMPLE 3) on day 0, 14 and 28. A blood sample was taken before vaccination (preimmune serum) and 29 days after the first immunization, which is hereafter referred as Q-Tcad1 serum.

B) Generation of Cell Lines Over Expressing T-Cadherin

T-cadherin cDNA was amplified by PCR from a differentiated C2C12 cells library and subcloned into the mammalian expression vector pGF (EMBO J. Saudan et al. 19 (16): 4351). This vector contains EGFP under the control of a retroviral LTR and the gene of interest (T-Cadherin) under the control of a CMV promoter. The resulting construct was named pGF-T-Cadherin. Based on the GFP expression cassette contained in this vector, transfected (T-Cadherin expressing) cells can easily be monitored by flow cytometry by monitoring the GFP expressing cells in the green channel (FL1) of the FACS machine. To generate cells which over express T-cadherin, 293-EBNA cells were transfected with pGF-T-Cadherin using lipofectamine 2000 according to the manufacturer's recommendation (Invitrogen). Two days after transfection the cells were collected and used for staining experiments as described below.

C) Analysis of T-Cadherin Specific Antibodies

The sera (preimmune serum and Qβ-Tacd1 serum) obtained above (A), were first analyzed for the presence of T-cadherin specific antibodies. Briefly, T-cadherin expressing cells (B), were stained with different dilutions of preimmune serum and Qb-Tacd1 serum in staining buffer (PBS supplemented with 1% FCS). The samples were then washed with staining buffer, stained with fluorescently labeled (Cy5) anti-mouse antibody, washed again with staining buffer and analyzed by FACS using a FACS Calibur (Becton Dickinson). The geometric mean fluorescence in the FL4 channel (Cy5) of the GFP positive population (FL1 channel, T-cadherin expressing cells) was determined. As shown in TABLE 2, a strong T-cadherin specific staining was observed with Qβ-Tacd1 serum whereas no shift in the geometric mean fluorescence was observed with preimmune serum. This result clearly shows that mice immunized with Qβ-Tacd1 produced antibodies that specifically bind to native T cadherin.

TABLE 2
Geometric mean fluorescence (GMFL) in the FL4 channel (Cy5)
of GFP/T-cadhcrin expressing cells stained with increasing
dilutions of preimmune serum or Qβ-Tcad1 serum.

GMFL

| | Preimmune I | Qβ-Tcad1 |
|---|---|---|
| dilution | Serum | Serum |
| 1:100 | 5 | 1315 |
| 1:200 | 5 | 987 |
| 1:400 | 4 | 667 |
| 1:800 | 4 | 414 |
| 1:1600 | 4 | 255 |

D) T-cadherin Specific Antibodies do not Interfere with the Binding Between T-Cadherin and Adiponectin

To test whether the T-cadherin specific antibodies inhibit the binding between T-cadherin and adiponectin, T-cadherin expressing cells obtained above (B) were first incubated with increasing dilutions of preimmune serum or Qβ-Tcad1 serum and then stained with a fixed concentration (1 μg/ml) of N-terminally FLAG tagged adiponectin (Alexis Biochemicals). Cells were then washed with staining buffer and adiponectin/T-cadherin binding was detected by staining with a Rabbit anti-FLAG antibody (SIGMA) followed by a staining with a fluorescently labeled (Cy5) anti-rabbit antibody (Jackson ImmunoResearch). Between the different incubation steps the samples were extensively washed with staining buffer. The stained samples were then analysed by FACS using a FACS Calibur (Becton Dickinson). The geometric mean fluorescence in the FM4 channel (Cy5) of the GFP positive population (FL1 channel, T-cadherin expressing cells) was determined. As shown in TABLE 3 in general the addition of high concentrations of serum (low dilutions, 1:5 or 1:10) unspecifically reduced adiponectin binding to the T-cadherin expressing cells as documented by a reduced geometric mean fluorescence in these samples compared to samples incubated with higher dilutions of sera (1:20, 1:40, 1:60, 1:80, 1:160, 1:320, 1:640). At every dilution tested, the geometric mean fluorescence obtained after incubation with preimmune serum was similar to the one observed with samples incubated with Qβ-Tcad1 serum, indicating that antibodies against T-cadherin in the Q-Tcad1 serum do not interfere with the binding of adiponectin to T-cadherin.

TABLE 3

GMFL

| | Preimmune | Qβ-Tcad1 |
|---|---|---|
| dilution | Serum | Serum |
| 1:5 | 79 | 79 |
| 1:10 | 97 | 118 |
| 1:20 | 158 | 192 |
| 1:40 | 189 | 179 |
| 1:80 | 202 | 196 |
| 1:160 | 219 | 236 |
| 1:320 | 203 | 221 |
| 1:640 | 210 | 228 |

### Example 15

### Testing of Immunisation Against T Cadherin to Prevent Angiogenesis Inhibition of Angiogenesis in the Chick Chorioallantoic Membrane (CAM) Assay

T-cadherin specific or control sera are generated by immunising mice or by chicken with either Qβ-Tcad1 or Qβ VLP as described in EXAMPLE 9. 35 days after the first immunisation, the mice are bled, serum is prepared and antibodies are purified using protein A (Pharmacia) according to the manufacturers recommendations. The purified antibodies are then used in a Chick Chorioallantoic Membrane (CAM) Assay. Briefly, after 3-d incubation at 37° C., fertilized white Leghorn eggs (OVA Production) are cracked, and chick embryos with intact yolks are carefully placed in 20-100-mm plastic Petri dishes. After 6 days of incubation in 4% CO2 at 37° C., methylcellulose disks containing 10, 30, 100, 300 or 900 mg of antibodies purified from T-cad1 immunised or Q-VLP immunised mice are implanted on the CAM of individual embryos. After 2-8 days of incubation, CAMs are examined for the formation of avascular zones around the field of the implanted disks using a stereoscope. The degree of vascularisation is compared between the samples that have been treated with antibodies purified from T-cad1 immunised mice and samples that have been treated with serum from Q-VLP immunised mice.

### Example 16

### In Vivo Angiogenesis Assay in Vaccinated Mice

Mice are vaccinated as described in EXAMPLE 9. 28 to 45 days after the first immunisation, a mouse corneal assay is performed as described previously (Cao, Y. & Cao, R. (1999) Nature 398, 381). Briefly, corneal micropockets are created with a modified von Graefe cataract knife in the eyes of 6 to 8-week-old C57BLy6J male mice. A micropellet (0.35×0.35 mm) of sucrose and aluminum sulphate coated with Hydron polymer type NCC (Interferon Sciences) containing 80 ng of FGF-2 is implanted into each pocket. The pellet is positioned 0.6-0.8 mm from the corneal limbus. After implantation, erythromycin ophthalmic ointment is applied to each eye. The eyes are examined by a slit-lamp biomicroscope on day 5 or day 6 after pellet implantation. Vessel length and clock-hours of circumferential neovascularization are measured in both, Qβ-Tcad1 and Qβ-VLP vaccinated animals.

### Example 17

### In Vivo Tumour Assay in Vaccinated Mice

Mice are vaccinated as described in EXAMPLE 9. 28 to 45 days after the first injection the effect of vaccination on tumour progression is tested in a model described previously (Eriksson, A. et al. (2002) Cancer Cell 1, 99-108). Briefly, murine T241 fibrosarcoma cells growing in log phase are harvested and resuspended in PBS, and 1×10⁶ cells in 100 μl are implanted s.c. in the middle dorsum C57BL6 female mice. Visible tumours are present after 72 h and measured using digital calipers at different time points. When the tumours have reached the size of the Swedish ethical upper limit (1.5 cm3) mice are killed, and the tumour tissues are removed and weighed. The size of the tumours in Qβ-Tcad1 and Qβ-VLP vaccinated animals are compared.

### Example 18

### The Effect of Antibodies Against T-Cadherin on Atherosclerosis

Monoclonal antibodies against T-cadherin are generated according to standard procedure in mice, using either full-length T-cadherin or T-cadherin domain 1 protein (expressed and purified as described in EXAMPLE 2) as immunogen. The resulting monoclonal antibodies are tested for their ability to interfere with the binding of adiponectin to T-cadherin as described in EXAMPLE 14. Seven to eight weeks old male Apoe^{-/-} mice (The Jackson Laboratory) are injected intraveneously with 100 μg of a monoclonal antibody selected from the above process every second week for 160 days. As a negative control, 5 mice are injected at the same regimen with an unrelated monoclonal control antibody. The mice are initially fed a normal chow diet, which is replaced after 3 weeks by a western diet (20% fat, 0.15% cholesterol, Provimi Kliba AG). 160 days after the first antibody injection, the mice are sacrificed and the aorta is isolated and prepared essentially as described (Tangirala R. K. et al. (1995) J. Lipd. Res. 36: 2320-2328), and the plaques are quantified with an imaging software (Motic Image Plus 2.0) on digital photographs. The plaque load is expressed as the sum of the surface of all plaques of the aorta taken up to the iliac bifurcation, divided by the total surface of the aorta measured up to the iliac bifurcation. The difference in median of the plaque load between the two groups of mice is analysed with a Mann-Whitney test, using a two-tailed p-value. The extent of atherosclerosis in each animal is further evaluated by histological analysis of cross-sections through the aortic origin, as described by Ludewig B. et al. (PNAS, (2000) 97:12752-12757).

Preferably the resulting monoclonal antibodies are tested for their ability to interfere with the binding of adiponectin to T-cadherin as described in EXAMPLE 14. Monoclonal antibodies that do not interfere with the binding are selected and used in the experiments as described above.

1. A composition comprising:
(a) a virus-like particle with at least one first attachment site; and
(b) at least one antigen with at least one second attachment site, wherein said at least one antigen is a T-cadherin domain protein, a combination of T-cadherin domain proteins, a T-cadherin domain fragment or a combination of T-cadherin domain fragments, wherein (a) and (b) are linked through said at least one first and said at least one second attachment site.

2. The composition of claim 1, wherein said at least one antigen is a T-cadherin domain protein, and wherein said T-cadherin domain protein is a T-cadherin domain 1 protein.

3. The composition of claim 1, wherein said at least one antigen is a T-cadherin domain protein, and wherein said T-cadherin domain protein comprises an amino acid sequence selected from the group consisting of:
(a) SEQ ID NO: 40;
(b) SEQ ID NO: 41;
(c) SEQ ID NO: 42;
(d) SEQ ID NO: 43;
(e) SEQ ID NO: 44; and
(f) an amino acid sequence which is at least 80%, preferably at least 80% identical with any one of SEQ ID NO: 40-44.

4. The composition of claim 1, wherein said virus-like particle comprises recombinant coat proteins, mutants or fragments thereof, of an RNA-bacteriophage.

5. The composition of claim 4, wherein said RNA-bacteriophage RNA-bacteriophage Qβ, fr, GA or AP205.

6. The composition of claim 1, wherein said first attachment site is linked to said second attachment site via at least one non-peptide covalent bond.

7. The composition of claim 1, wherein said first attachment site comprises an amino group.

8. The composition of claim 1, wherein said second attachment site comprises a sulfhydryl group.

9. (canceled)

10. The composition of claim 1, wherein said virus-like particle is recombinantly produced in a host and wherein said virus-like particle is essentially free of host RNA, preferably host nucleic acids.

11. The composition of claim 10 further comprising at least one polyanionic macromolecule, wherein said polyanionic macromolecule is packaged in said virus-like particle.

12. The composition of claim 11, wherein said at least one polyanionic macromolecule is polyglutamic acid and/or polyaspartic acid.

13. (canceled)

14. (canceled)

15. A method of immunization comprising administering said composition of claim 1 to an animal or a human.

16. A pharmaceutical composition comprising
(a) the composition of claim 1; and
(b) a pharmaceutically acceptable carrier.

17. (canceled)

18. (canceled)

19. (canceled)

20. (canceled)

21. A method of treating a disease in an animal or a human comprising administering said composition of claim 1 to said animal or human, wherein said disease is selected form the group consisting of coronary artery disease, atherosclerosis, obesity, type I diabetes, type II diabetes and cancer.

22. A method of treating a disease in an animal or a human comprising administering at least one substance to said animal or human, wherein said substance is characterized by being capable of binding to T-cadherin and wherein said disease is selected form the group consisting of coronary artery disease, atherosclerosis, obesity, type I diabetes, type II diabetes and cancer.

23. The method of claim 22, wherein said substance is an antibody specifically binding to T-cadherin; and wherein preferably said antibody is produced in response to the composition of claim 1.

24. (canceled)

25. The method of claim 22, wherein said disease is atherosclerosis.

26. The composition of claim 1, wherein said virus-like particle is a virus-like particle of RNA-bacteriophage.

27. The composition of claim 1, wherein said virus-like particle comprises recombinant coat proteins of RNA-bacteriophage Qβ, wherein said coat proteins consist of the amino acid sequence as set forth in SEQ ID NO:1.

28. The composition of claim 1, wherein said virus-like particle is a virus-like particle of RNA-bacteriophage AP205, wherein said antigen is a T-cadherin domain fragment, and wherein said virus-like particle comprises coat proteins, or mutants thereof, of RNA-bacteriophage AP205, and wherein said antigen is linked via either the N- or the C-terminus of said coat protein, or mutant thereof, by way of at least one peptide bond.


SEQUENCE LISTING (61 sequences)

SEQ ID NO: 1 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 2 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 3 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 4 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 5 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 6 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 7 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 8 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 9 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 10 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 11 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 12 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 13 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 14 - protein, 131 aa, bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 15 - protein, 132 aa, Artificial Sequence
  Synthetic Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 16 - protein, 132 aa, Artificial Sequence
  Synthetic Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 17 - protein, 132 aa, Artificial Sequence
  Synthetic Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Synthetic Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Synthetic Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 21 - protein, 188 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 22 - protein, 714 aa, Mus musculus
MQPRTPLTLCVLLSQVLLVTSADDLECTPGFQRKVLHIHQPAEFIEDQPVLNLTFNDCKGNEKLHYEVSSPHFKVNSDGTLVALRNITAVGRTLFVHARTPHAEDMAELVIVGGKDIQGSLQDIFKFARTSPVPRQKRSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFREGPYIGHVMEGSPTGTTVMRMTAFDADDPATDNALWRYNIRQQTPDKPSPNMFYIDPEKGDIVTVVSPALLDRETLENPKYELIIEAQDMAGLDVGLTGTATATIVIDDKNDHSPKFTKKEFQARVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTKQENISVGSVLLTVNATDPDSLQHQTIRYSIYKDPAGWLSINPINGTVDTTAVLDRESPFVHNSVYTALFLAIDSGNPPATGTGTLLITLEDINDNAPVIYPTVAEVCDDARNLSVVILGASDKDLHPNTDPFKFEIHKQTVPDKVWKISKINNTHALVSLLQNLNKANYNLPIMVTDSGKPPMTNITDLKVQVCSCKNSKVDCNGAGALHLSLSLLLLFSLLSLLSGL

SEQ ID NO: 23 - protein, 563 aa, Mus musculus
SPVPRQKRSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFREGPYIGHVMEGSPTGTTVMRMTAFDADDPATDNALWRYNIRQQTPDKPSPNMFYIDPEKGDIVTVVSPALLDRETLENPKYELIIEAQDMAGLDVGLTGTATATIVIDDKNDHSPKFTKKEFQARVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTKQENISVGSVLLTVNATDPDSLQHQTIRYSIYKDPAGWLSINPINGTVDTTAVLDRESPFVHNSVYTALFLAIDSGNPPATGTGTLLITLEDINDNAPVIYPTVAEVCDDARNLSVVILGASDKDLHPNTDPFKFEIHKQTVPDKVWKISKINNTHALVSLLQNLNKANYNLPIMVTDSGKPPMTNITDLKVQVCSCKNSKVDCNGAG

SEQ ID NO: 24 - protein, 103 aa, Mus musculus
SPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIF

SEQ ID NO: 25 - protein, 118 aa, Mus musculus
REGPYIGHVMEGSPTGTTVMRMTAFDADDPATDNALWRYNIRQQTPDKPSPNMFYIDPEKGDIVTVVSPALLDRETLENPKYELIIEAQDMAGLDVGLTGTATATIVIDDKNDHSPKF

SEQ ID NO: 26 - protein, 114 aa, Mus musculus
TKKEFQARVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVF

SEQ ID NO: 27 - protein, 108 aa, Mus musculus
YPDPMMVTKQENISVGSVLLTVNATDPDSLQHQTIRYSIYKDPAGWLSINPINGTVDTTAVLDRESPFVHNSVYTALFLAIDSGNPPATGTGTLLITLEDINDNAPVI

SEQ ID NO: 28 - protein, 95 aa, Mus musculus
YPTVAEVCDDARNLSVVILGASDKDLHPNTDPFKFEIHKQTVPDKVWKISKINNTHALVSLLQNLNKANYNLPIMVTDSGKPPMTNITDLKVQVC

SEQ ID NO: 29 - DNA, 28 bp, artificial sequence
  Synthetic forward primer cloning CAD1
ctagctagctccattgtggtgtccccca

SEQ ID NO: 30 - DNA, 28 bp, artificial sequence
  Synthetic primer to clone CAD1 reverse
ctactcgaggaagatgggtctgttgtcg

SEQ ID NO: 31 - DNA, 28 bp, artificial sequence
  Synthetic primer to clone CAD2 FWD
ctagctagccgggaaggcccttacatcg

SEQ ID NO: 32 - DNA, 33 bp, artificial sequence
  Synthetic primer to clone CAD2 REV
ctactcgaggaattttggtgagtgatcattttt

SEQ ID NO: 33 - DNA, 31 bp, artificial sequence
  Synthetic primer to clone CAD3 FWD
ctagctagcaccaagaaagagtttcaagcca

SEQ ID NO: 34 - DNA, 29 bp, artificial sequence
  Synthetic primer to clone CAD3 REV
ctactcgaggaagactggtccctcgttga

SEQ ID NO: 35 - DNA, 28 bp, artificial sequence
  Synthetic primer to clone CAD4 FWD
ctagctagctacccggaccccatgatgg

SEQ ID NO: 36 - DNA, 31 bp, artificial sequence
  Synthetic primer to clone CAD4 REV
ctactcgagaatgacaggagcgttgtcatta

SEQ ID NO: 37 - DNA, 29 bp, artificial sequence
  Synthetic primer to clone CAD5 FWD
ctagctagctacccaactgtggctgaggt

SEQ ID NO: 38 - DNA, 29 bp, artificial sequence
  Synthetic primer to clone CAD5 REV
ctactcgagagagcacacctgcaccttga

SEQ ID NO: 39 - protein, 3823 aa, homo sapiens
AGCGGCTCTCCTCAAAGCCTGGCTCCCACGGAAAATATGCTCAGTGCAGCCGCGTGCATGAATGAAAACGCCGCCGGGCGCTTCTAGTCGGACAAAATGCAGCCGAGAACTCCGCTCGTTCTGTGCGTTCTCCTGTCCCAGGTGCTGCTGCTAACATCTGCAGAAGATTTGGACTGCACTCCTGGATTTCAGCAGAAAGTGTTCCATATCAATCAGCCAGCTGAATTCATTGAGGACCAGTCAATTCTAAACTTGACCTTCAGTGACTGTAAGGGAAACGACAAGCTACGCTATGAGGTCTCGAGCCCATACTTCAAGGTGAACAGCGATGGCGGCTTAGTTGCTCTGAGAAACATAACTGCAGTGGGCAAAACTCTGTTCGTCCATGCACGGACCCCCCATGCGGAAGATATGGCAGAACTCGTGATTGTCGGGGGGAAAGACATCCAGGGCTCCTTGCAGGATATATTTAAATTTGCAAGAACTTCTCCTGTCCCAAGACAAAAGAGGTCCATTGTGGTATCTCCCATTTTAATTCCAGAGAATCAGAGACAGCCTTTCCCAAGAGATGTTGGCAAGGTAGTCGATAGTGACAGGCCAGAAAGGTCCAAGTTCCGGCTCACTGGAAAGGGAGTGGATCAAGAGCCTAAAGGAATTTTCAGAATCAATGAGAACACAGGGAGCGTCTCCATGACACGGACCTTGGACAGAGAAGTAATCGCTGTTTATCAACTATTTGTGGAGACCACTGATGTCAATGGCAAAACTCTCGAGGGGCCGGTGCCTCTGGAAGTCATTGTGATTGATCAGAATGACAACCGACCGATCTTTCGGGAAGGCCCCTACATCGGCCACGTCATGGAAGGGTCACCCACAGGCACCACAGTGATGCGGATGACAGCCTTTGATGCAGATGACCCAGCCACCGATAATGCCCTCCTGCGGTATAATATCCGTCGGCAGACGCCTGACAAGCCATCTCCCAACATGTTCTACATCGATCCTGAGAAAGGAGACATTGTCACTGTTGTGTCACCTGCGCTGCTGGACCGAGAGACTCTGGAAAATCCCAAGTATGAACTGATCATCGAGGCTCAAGATATGGCTGGACTGGATGTTGGATTAACAGGCACGGCCACAGCCACGATCATGATCGATGACAAAAATGATCACTCACCAAAATTCACCAAGAAAGAGTTTCAAGCCACAGTCGAGGAAGGAGCTGTGGGAGTTATTGTCAATTTGACAGTTGAAGATAAGGATGACCCCGCCACAGGTGCATGGAGGGCTGCCTACACCATCATCAACGGAAACCCCGGGCAGAGCTTTGAAATCCACACCAACCCTCAAACCAACGAAGGGATGCTTTCTGTTGTCAAACCATTGGACTATGAAATTTCTGCCTTCCACACCCTGCTGATCAAAGTGGAAAATGAAGACCCACTCGTACCCGACGTCTCCTACGGCCCCAGCTCCACAGCCACCGTCCACATCACTGTCCTGGATGTCAACGAGGGCCCAGTCTTCTACCCAGACCCCATGATGGTGACCAGGCAGGAGGACCTCTCTGTGGGCAGCGTGCTGCTGACAGTGAATGCCACGGACCCCGACTCCCTGCAGCATCAAACCATCAGGTATTCTGTTTACAAGGACCCAGCAGGTTGGCTGAATATTAACCCCATCAATGGGACTGTTGACACCACAGCTGTGCTGGACCGTGAGTCCACATTTGTCGACAACAGCGTGTACACTGCTCTCTTCCTGGCAATTGACAGTGGCAACCCTCCCGCTACGGGCACTGGGACTTTGCTGATAACCCTGGAGGACGTGAATGACAATGCCCCGTTCATTTACCCCACAGTAGCTGAAGTCTGTGATGATGCCAAAAACCTCAGTGTAGTCATTTTGGGAGCATCAGATAAGGATCTTCACCCGAATACAGATCCTTTCAAATTTGAAATCCACAAACAAGCTGTTCCTGATAAAGTCTGGAAGATCTCCAAGATCAACAATACACACGCCCTGGTAAGCCTTCTTCAAAATCTGAACAAAGCAAACTACAACCTGCCCATCATGGTGACAGATTCAGGGAAACCACCCATGACGAATATCACAGATCTCAGGGTACAAGTGTGCTCCTGCAGGAATTCCAAAGTGGACTGCAACGCGGCGGGGGCCCTGCGCTTCAGCCTGCCCTCAGTCCTGCTCCTCAGCCTCTTCAGCTTAGCTTGTCTGTGAGAACTCCTGACGTCTGAAGCTTGACTCCCAAGTTTCCATAGCAACAGGAAAAAAAAAAATCTATCCAAATCTGAAGATTGCGGTTTACAGCTATCGAACTTCACAACTAGGCCTCAATTGTTCCGGTTTTTTATTTTCTTTACAATTTCACTTAGTCTGTACTTCATCATTTTGACAGCATCTTCCTCCCTCCTTTAATTAATGGAATCTTCTGAATTTTCCCTGAATGTTTAAAGATCATGACATATGACTTGATCTTCTGGGAGCAGGAACAATGACTACTTTTTCTGGTGTGTTAACATGTCGCTAGCCAGTGCTCCAGGCACCCAGCTTTGTCTGTGGGTTAGTATTGGTGTATGTATGAGTATCTGTATGTATATATACACGGTATTTATAGAGAGAGACTATCCTGGAGAAGCCTCGTTTTGATGCCATTCTTCCTTGCAAGGTTAAGCAAGGTGGGTGGAAACTAAGACACCTGAACCCTCCAGGGCCTCCCGCATCAAGGTCAGCATGAGGACAGACCACAGAGCTGTCACTTTTGCTCCGAAGCTACTTCTCCACTGTCCCGTTCAGTCTGAATGCTGCCACAACCAGCCAGGCAGGTCCACAGAGAGGGAGAGCAGAGAAAGAAGTCCTTTCTCTTTATTGAGTTCGAGGACTACAACCAATTTACACTGCCATCTGATGCCGTGATCCTGAGCCAAGGAGGTGAGGAGCAGAGCAGGCAATTTCACCACCAAATGCCAAGAAAAGGGCTGACATTTTCTTTCATGGGCACCAACCTGCATTTGTATGTGTCCCGAATCCACAGTCGTACTGATTCTAATGGGGACACAGATCATGGTAGAGAATCTCTCCCTCCTCAGTAAATGTACAACTGCACCTGTCATCATGGAGGTCATACATGCATACAAAGAGGTGTACAGGTACCATCTTGTATACACATATATACCCACATGTACAGACATACATTTATGCGCATTCACGCTGTTTGTTTCATATATACAGGCATAAAATAGAGTAAATACAGGTAGTTTTAAAAGTACCCTTTTGTGTGAATTGACTACCGTTGTTTGCAAACCCGAAAATAAAAGACGTTCATTATGTATGAAAAGTAACTGATTTGTATTCTGTGAGCATGTAAAAGCGGAAAGTTAGTGCTTGTTTTAAGATTACCTTCTTGTTGATAAACCATAAATGAATCATCAAAGCTCACACCAAATTTTTCTATCAAATAAAACTAGTGACAGCTTGTGGCTTTTTATTAGAGCTCGCCACGAACTAGGGTAAGGTGAGTGTCTTAGCATATTTTAATGCAGTTGCTTACTAAAGGTTTTAACCGCACATGCACACACACACGCTTTCTTATGCAATCTATGTTTGCACTTGTGCTTTCAGTTAGCCTTCTGTAGGAAGTAGAAGTCATATGTTGTCTTTGTTGTAGTGAAATTATACAGATAGAGTTCCATATATTGTATTTGTTTCAATGGTAAATCCTTTTGGAACATATAGAATGCAGAGATTTTTTTTTCCATTAAAATAAATGGGTATTGGTGGTTAAAACTGAAAAAAAAAAAAAAAA

SEQ ID NO: 40 - protein, 103 aa, homo sapiens
SPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIF

SEQ ID NO: 41 - protein, 118 aa, homo sapiens
REGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKF

SEQ ID NO: 42 - protein, 114 aa, homo  sapiens
TKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVF

SEQ ID NO: 43 - protein, 108 aa, homo sapiens
YPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFI

SEQ ID NO: 44 - protein, 95 aa, homo sapiens
YPTVAEVCDDAKNLSVVILGASDKDLHPNTDPFKFEIHKQAVPDKVWKISKINNTHALVSLLQNLNKANYNLPIMVTDSGKPPMTNITDLRVQVC

SEQ ID NO: 45 - protein, 713 aa, homo sapiens
MQPRTPLVLCVLLSQVLLLTSAEDLDCTPGFQQKVFHINQPAEFIEDQSILNLTFSDCKGNDKLRYEVSSPYFKVNSDGGLVALRNITAVGKTLFVHARTPHAEDMAELVIVGGKDIQGSLQDIFKFARTSPVPRQKRSIVVSPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAGALRFSLPSVLLLSLFSLACL

SEQ ID NO: 46 - protein, 161 aa, artificial sequence
  Synthetic CAD1 sequence expressed from pMODC6
MDPHHHHHHGSGDDDDKALVGCGGPKPSTPPGSSGGAPASSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFLEFKRGRMHHHHHH

SEQ ID NO: 47 - protein, 555 aa, homo sapiens
SIVVSPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAG

SEQ ID NO: 48 - protein, 10 aa, artificial sequence
  Synthetic gamma linker 1
CGDKTHTSPP

SEQ ID NO: 49 - protein, 6 aa, artificial sequence
  Synthetic N terminial glycine linker
GCGGGG

SEQ ID NO: 50 - protein, 6 aa, artificial sequence
  Synthetic N-terminal glycine serine linker
CGGGGS

SEQ ID NO: 51 - protein, 9 aa, artificial sequence
  Synthetic GCGSGGGGS linker
GCGSGGGGS

SEQ ID NO: 52 - protein, 10 aa, artificial sequence
  Synthetic C-terminal gamma 1 linker
DKTHTSPPCG

SEQ ID NO: 53 - protein, 18 aa, artificial sequence
  Synthetic C terminal gamma linker 3
PKPSTPPGSSGGAPGGCG

SEQ ID NO: 54 - protein, 6 aa, artificial sequence
  Synthetic C terminal glycine linker
GGGGCG

SEQ ID NO: 55 - protein, 6 aa, artificial sequence
  Synthetic C terminal glycine serine linker
SGGGGC

SEQ ID NO: 56 - protein, 10 aa, artificial sequence
  Synthetic GSGGGGSGCG linker
GSGGGGSGCG

SEQ ID NO: 57 - protein, 6 aa, artificial sequence
  Synthetic glycine lysine linker
GGKKGC

SEQ ID NO: 58 - protein, 6 aa, artificial sequence
  Synthetic glycine lysine linker 2
CGKKGG

SEQ ID NO: 59 - protein, 17 aa, artificial sequence
  Synthetic CGGPKPSTPPGSSGGAP
CGGPKPSTPPGSSGGAP

SEQ ID NO: 60 - protein, 7 aa, artificial sequence
  Synthetic T-cad conserved sequence in domain 1
INENTGS

SEQ ID NO: 61 - protein, 5 aa, artificial sequence
  Synthetic T-cad conserved sequence in domain 1
ETTDV